(19) United States
(12) Patent Application Publication
(10) Pub. No.: US 2004/0228860 A1
(43) Pub. Date: Nov. 18, 2004

(54) Antibodies to a tumor-associated surface antigen for delivery of diagnostic and therapeutic agents

(75) Inventors: Ronald Taylor, Charlottesville, VA (US); Alessandra Nardin, Paris (FR); William M. Sutherland, Earlysville, VA (US); Mitchell H. Sokoloff, Hinsdale, IL (US); Leland Chung, Lovingston, VA (US)

Correspondence Address: JONES DAY, 222 EAST 41ST ST, NEW YORK, NY, 10017, US

(73) Assignee: The University of Virginia Patent Foundation

(21) Appl. No.: 10/720,019

(22) Filed: Nov. 21, 2003

Related U.S. Application Data
(63) Continuation of application No. 09/392,500, filed on Sep. 9, 1999, now abandoned.
(60) Provisional application No. 60/099,782, filed on Sep. 10, 1998.
(60) Provisional application No. 60/123,786, filed on Mar. 11, 1999.

(51) Int. Cl. A61K 39/395

(52) U.S. Cl. 424/144.1; 424/178.1

(57) ABSTRACT

The present invention relates to the treatment, inhibition and prevention of cancer by the administration of anti-C3b(i) antibodies. The invention also relates to the treatment, inhibition and prevention of cancer by the administration of IgM antibodies and/or complement components prior to the administration of anti-C3b(i) antibodies. The present invention further relates to the detection, imaging, diagnosis and monitoring of cancer utilizing C3b(i) specific antibodies.

This application is entitled to and claims priority benefits of application No. 60/099,782 filed Sep. 10, 1998 and application No. 60/123,786 filed Mar. 11, 1999, the entire disclosures of which are incorporated herein by reference.

This invention was made, in part, with government support under Grant Number AR43307 awarded by the National Institutes of Health. The United States government has certain rights in the invention.

### 1. INTRODUCTION

The present invention relates to methods of treatment, inhibition and prevention of cancer by the administration of antibodies specific for C3b(i). The present invention also relates to the treatment and prevention of cancer by administering IgM antibodies and/or complement components prior to the administration of antibodies specific for C3b(i). The present invention further relates to pharmaceutical compositions comprising antibodies specific for C3b(i). Further, the present invention relates to the detection, imaging, and diagnosis of cancer utilizing antibodies specific for C3b(i).

### 2. BACKGROUND OF THE INVENTION

Despite advances in prevention and early detection, refinements in surgical technique, and improvements in adjuvant radio- and chemotherapy, the ability to cure many patients of cancer remains elusive. This is especially pertinent to prostate cancer, which remains the most prevalent visceral tumor in American men, with approximately 180,000 new cases and nearly 40,000 deaths expected in 1999 (Landis et al., 1999, Cancer J Clin 49: 8-31). The continuing challenge of prostate cancer treatment is the successful management and eradication of recurrent, metastatic, and hormone-refractory disease, which accounts for the vast majority of prostate cancer-specific morbidity and mortality (Small, 1998, Drugs and Aging 13:71-81).

Many treatment modalities currently under investigation for prostate and other cancers depend upon tissue-specific delivery of anti-neoplastic agents. One immunotherapeutic approach involves conjugating cytotoxic agents to monoclonal antibodies (mAbs) specific for a particular cancer cell epitope. In this manner, the therapeutic agents can be delivered at a high therapeutic dose directly, and selectively, to the tumor site, thereby minimizing injury to healthy tissue (Bach et al., 1993, Immunol Today 14:421-5; Reithmuller et al., 1993, Cur Op Immunol 5:732-9; Gruber et al., 1996, Spring Sem Immunopath 18:243-51). This method first requires the identification of specific epitopes for each cancer type. Such candidate epitopes must be expressed at high levels on the cancer cells compared to normal tissue. Second, this method requires the development of high affinity mAbs specific for these epitopes and these mAbs must show minimal cross-reactivity with self tissue. The biological mechanism of killing with mAbs will be variable, depending upon the epitopes identified on the cancer cells, and the effector functions of the specific mAb isotype. However, due to antigenic modulation and/or mutation, the cancer cells may reduce the available levels of the target epitope per cell, or eliminate it from their surface altogether. Thus, the use of mAbs in cancer diagnosis and treatment remains problematic.

A more widely applicable approach to treatment of cancer with mAbs would be to identify a ubiquitous antigenic site, present on virtually all cancer cells, and then to develop a panel of mAbs specific for this antigen. A voluminous literature reveals that cancer cells share certain common characteristics. Many types of human cancer cells are characterized by substantial abnormalities in the glycosylation patterns of their cell-surface proteins and lipids (Hakomori et. al., 1996, Canc Res. 56:5309-18; Castronovo et al.,1989, J Nat Canc Inst 81:212-6; Springer et al.,1984, Science 224:1198-206; Springer et al., 1997, J Mol Med 75:594-602). These differences have led to the identification of antigenic determinants on cancer cells which are expressed at far lower levels on normal cells. Natural IgM antibodies to these epitopes are present in the circulation, and the interaction of such IgM antibodies with these cancer cell surface antigens leads to activation of complement and covalent coupling of complement activation products (C3b and its fragments, collectively referred to as C3b(i)) to the tumor cells (Okada et al., 1974, Nature 248:521-25; Irie et. al., 1974, Science 186:454-456; Desai et al., 1995, J Immunol Methods 188:175-85; Vetvicka et al., 1996, J Clin Invest 98:50-61; Vetvicka et al., 1997, J Immunol 159:599-605; Vetvicka et al., 1999, Clin Exp Immunol 115:229-35). Although relatively large amounts of C3b(i) can be deposited on cancer cells, the concomitant expression of high levels of membrane-associated complement control proteins (e.g., decay accelerating factor (“DAF”), membrane cofactor protein (“MCP”), and, in particular, “protectin” i.e., CD59) usually prevents complement-mediated lysis (Cheung et al., 1988, J Clin Invest 81:1122-8; Gorter et al., 1996, Lab Invest 74:1039-49; Maenpaa et al., 1996, Am J Path 148:1139-52; Li et al.,1997, Int J Canc 71:1049-55). Further, several investigators have established that in most cases, cancer patients have substantially lowered levels of the potentially protective IgM antibodies. Thus, in many cases the cancer cells cannot easily be killed by complement activation because of the reduced levels of protective IgM antibody and the increased expression of human complement control proteins on their surface.

2.1 The Complement System

The complement system which is composed of some 21 plasma proteins plays an important role in the human immune system, both in the resistance to infections and in the pathogenesis of tissue injury. The activated products of the complement system attract phagocytic cells and greatly facilitate the uptake and destruction of foreign particles by opsonization. There are two distinct pathways for activating complement, the classical pathway and the alternate pathway, that result in conversion of C3 to C3b and subsequent responses (e.g., the formation of the membrane attack complex (“MAC”)). Activation of the classical pathway is initiated by antigen-antibody complexes or by antibody bound to cellular or particulate antigens. The alternate pathway is activated independent of antibody by complex polysaccharides in pathogens such as bacterial wall constituents, bacterial lipopolysaccharides (LPS), cell wall constituents of yeast (zymosan).

The classic complement pathway is initiated by the binding of C1 to immune complexes containing IgG or IgM antibodies. Activated C1 cleaves C2 and C4 into active components, C2a and C4b. The C4b2a complex is an active protease called C3 convertase, and acts to cleave C3 into C3a and C3b. C3b forms a complex with C4b2a to produce C4b2a3b, which cleaves C5 into C5a and C5b. C5b combines with C6, and the C5b6 complex combines with C7 to form the ternary complex C5b67. The C5b67 complex binds C8 to form the C5b678 complex which in turn binds C9 and results in the generation of the C5-C9 MAC. The insertion of the MAC into the cell membrane results the formation of a transmembrane channel that causes cell lysis.

In the alternative pathway, conversion of C3 to C3b (or C3i) produces a product that can combine with factor B, giving C3bB (or C31B). These complexes are acted upon by factor D to generate C3bBb, which is a C3 convertase capable of cleaving more C3 to C3b, leading to more C3bBb and even more C3 conversion. Under certain circumstances the C3bBb complex is stabilized by association with the positive regulator properdin (P) by association of C3b and Bb. The C3 convertases can associate with an additional C3b subunit to form the C5 convertase, C3bBbC3b, which is active in the production of the C5-C9 MAC.

In both the classical and alternative pathways, the critical step in the activation of complement is the proteolytic conversion of C3 to the fragments C3b and C3a. C3a is an anaphylatoxin that attracts mast cells to the site of challenge, resulting in local release of histamine, vasodilation and other inflammatory effects. The nascent C3b has an ability to bind to surfaces around its site of generation and functions as a ligand for C3 receptors mediating, for example, phagocytosis.

Endogenous cell surfaces normally exposed to complement are protected by membrane-bound regulators such as decay accelerating factor (“DAF”), C59 (“protectin”), MCP, and the soluble C1 inhibitor or C1NH. DAF and MCP are responsible for limiting production of C3b and insure the generation of inactive forms of C3b, C3bi and C3dg from C3b. CD59 prevents attack of the MAC, which would otherwise destroy the cancer cell. C1 inhibitor binds to the active subcomponents of C1, C1r and C1s, and inhibits their activity.

Citation of a reference in this section or any section of this application shall not be construed as an admission that such reference is prior art to the present invention.

### 3. SUMMARY OF THE INVENTION

The present invention encompasses methods, compounds and compositions for the treatment and prevention of cancer by the administration of antibodies specific for C3b(i). The term “C3b(i)” as used herein refers to C3b and its fragments. The present invention also encompasses methods, compounds and compositions for the treatment, inhibition and prevention of cancer by the enrichment of IgM antibodies and/or complement components prior to the administration of native or recombinant anti-C3b(i) antibodies or fragments thereof. The present invention encompasses methods of depleting cancerous cells in vitro utilizing antibodies or fragment thereof specific for C3b(i). Further, the present invention encompasses methods and kits for the detection, imaging, and diagnosis of cancer utilizing antibodies or fragments thereof specific for C3b(i).

The present invention provides a method for treating or preventing cancer in a subject comprising administering to the subject an amount of antibody to C3b(i) or an antibody to C3b(i) covalently linked to a second molecule (e.g., an IgM antibody, a glycoprotein or a glycolipid), effective to treat or prevent cancer. The invention provides a method for treating or preventing cancer in a subject comprising administering to the subject an amount of a nucleic acid sequence encoding an antibody to C3b(i) or an antibody to C3b(i) covalently linked to a second molecule, effective to treat or prevent cancer. The present invention provides a method for treating or preventing cancer in a subject comprising administering to the subject an amount of an antibody to C3b(i) or an antibody to C3b(i) covalently linked to a second molecule and IgM antibody, effective to treat or prevent cancer. The present invention provides a method for treating or preventing cancer in a subject comprising administering to the subject an amount of an antibody to C3b(i) or an antibody to C3b(i) covalently linked to a second molecule and one or more complement components, effective to treat or prevent cancer. The present invention also provides a method for treating or preventing cancer in a subject comprising administering to the subject an amount of an antibody to C3b(i) or an antibody to C3b(i) covalently linked to a second molecule, IgM antibody and one or more complement components, effective to treat or prevent cancer. The present invention further provides a method of depleting cancer cells from cells obtained from an animal with cancer comprising contacting in vitro a sample comprising cells obtained from said animal with antibody to C3b(i) or an antibody to C3b(i) covalently linked to a second molecule.

The present invention provides a pharmaceutical composition comprising an antibody to C3b(i) or an antibody to C3b(i) covalently linked to a second molecule, in an amount effective to inhibit or prevent cancer in a subject. The invention provides a pharmaceutical composition comprising nucleic acid encoding an antibody to C3b(i) or an antibody to C3b(i) covalently linked to a second molecule, in an amount effective to inhibit or prevent cancer in a subject. The present invention further provides a pharmaceutical composition comprising a bispecific antibody which is specific for C3b(i) or C3b(i) covalently linked to a second molecule and an effector cell receptor or antigen, in an amount effective to inhibit or present cancer in a subject.

The present invention provides a method for detecting cancer comprising: a) administering to a subject an effective amount of a labeled antibody which specifically binds to C3b(i) or a labeled antibody which specifically binds to C3b(i) covalently linked to a second molecule; b) waiting for a time interval following the administering to permit the labeled antibody to preferentially concentrate at any cancerous site in the subject; c) determining background level; and d) detecting the labeled antibody in the subject, wherein detection of the labeled antibody above the background level indicates the presence of a cancer. The present invention also provides a method for detecting cancer in a subject, comprising imaging said subject at a time interval after administration to said subject of an effective amount of a labeled antibody which specifically binds to C3b(i) or which specifically binds to C3b(i) covalently linked to a second molecule, said time interval being sufficient to permit the labeled antibody to preferentially concentrate at any cancerous site in said subject, wherein detection of the labeled antibody localized at said site in the subject indicates the presence of cancer.

The invention provides a kit comprising, in one or more containers, an antibody to C3b(i) or an antibody to C3b(i) covalently linked to a second molecule.

The present invention further encompass methods, compounds and compositions for the treatment and prevention of cancer by the administration of IgM antibodies and/or one or more complement components without antibodies to C3b(i) or antibodies to C3b(i) covalently linked to a second molecule.

Reference is made herein to antibody specific for C3b(i), or C3b(i) specific antibodies, or anti-C3b(i) antibodies and the like; as used herein such reference shall also be construed as reference to an antibody to C3b(i) covalently linked to a second molecule, unless indicated otherwise explicitly or by context.

### 4. BRIEF DESCRIPTION OF THE FIGURES

FIG. 1 (A-D). Representative flow cytometry data from a study with serum from a normal donor (A, B) and a cancer patient (C, D). Measurement of C3b(i) (A, C) and IgM (B, D) deposition on C4-2 human prostate cancer cells is shown. Abundant C3b(i) is deposited on C4-2 cancer cells in response to the addition of normal human serum; this opsonization appears to be facilitated by both the classical and alternative complement pathways. After opsonization with serum from a prostate cancer patient, significantly less C3b(i) and IgM are deposited on the tumor cells (C, D). C3b(i) deposition via the alternative pathway (serum with Mg-EGTA), however, is comparable for both the normal and cancer patient serum, suggesting that the alternative pathway of the complement system remains intact in prostate cancer patient serum.

FIG. 2 (A-B). Flow cytometry and radioimmunoassay data demonstrating that removal of IgM results in a large reduction in the amount of C3b(i) that is deposited on LNCap (A) or C4-2 (B) cells. Normal C3b(i) deposition can be restored with either whole normal human plasma (A, B) (e.g., plasma/IgM-depleted serum), which provides a source of human IgM, or with purified IgM (B).

FIG. 3 (A-B). Radioimmunoassay data demonstrating that the classical pathway of complement activation generates between 20,000 and 70,000 C3b(i) epitopes per C4-2 cell, as defined by binding of both ¹²⁵I-labeled mAbs 8E11(A) and 7C12 (B). C3b(i) deposition is dependent upon the amount of serum used (low=50% NHS in T-media; high=75% NHS in T-media).

FIG. 4. Flow cytometry results from surveys of sera from normal donors and patients with prostate cancer. Binding of human immunoglobulin to LNCaP and C4-2 prostate cancer cells was measured. Significant differences were determined by t tests.

FIG. 5 (A-B). Immunohistochemical staining of normal and neoplastic human prostate tissue after incubation with anti-C3b(i) mAbs.

FIG. 6. Rosetting experiment using erythrocytes and opsonized C4-2 prostate cancer cells in the presence of a cocktail of anti-C3b(i) X anti-CR1 bispecific mAb complexes (7C12 X 1B4 and 7C12 X 9H3). The incubations were performed in plasma.

FIG. 7 (A-B). In vitro killing of LNCaP (A) and C4-2 (B) prostate cancer cells using ¹³¹I-labeled mAbs. Dashed line (----) delineates normal serum opsonized cells treated with ¹³¹I-labeled irrelevant mAbs; dotted line (....) delineates non-opsonized cells treated with ¹³¹I-anti-Cb3(i) mAbs; solid line (—) delineates normal serum opsonized cells treated with ¹³¹I-labeled anti-C3b(i) mAbs. Measured as cell proliferation relative to non-treated cells.

FIG. 8. The schematic illustrates the steps of the invention, all of which occur on the cell surface of tumor cells within the body of the cancer patient. In the first step, human IgM (either endogenous, or infused into the patient) binds to specific sites on the cancer cell. In the second step, complement (either endogenous, or infused into the patient as fresh plasma) is activated, and the resulting proteolytic fragment C3b(i) is deposited on the surface of the cancer cell. In the third step, a mAb specific for the C3b(i) epitope is administered. The mAb can be associated with a toxic, enzymatic, genetic, differentiating, and/or imaging agent (therefore it is an “effector mAb”), which results in the destruction or imaging of the cancer cell.

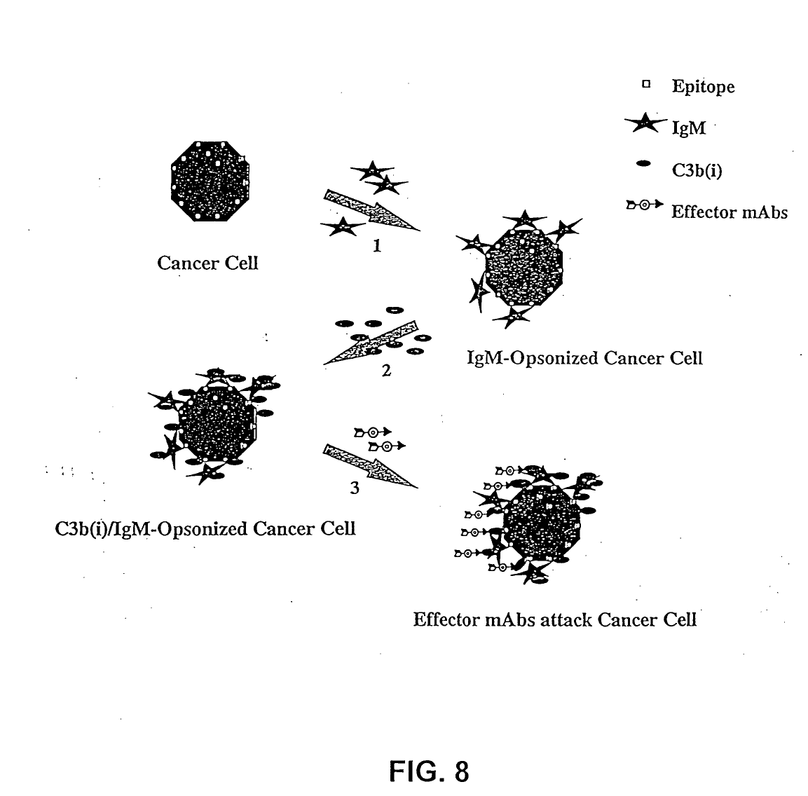

### 5. DETAILED DESCRIPTION OF THE INVENTION

The present invention encompasses compositions and methods of treatment, inhibition and prevention of cancer by the administration of C3b(i) specific antibodies. The present invention also encompasses compositions and methods of treatment, inhibition and prevention of cancer by administering of IgM and/or one or more complement components prior to the administration of C3b(i) specific antibodies. In particular, the present invention encompasses compositions and methods of treatment or inhibition of maligancies or proliferative disorders including, but not limited to, leukemia, polycythemia vera, lymphoma (e.g., Hodgkin's disease and non-Hodgkin's disease), multiple myeloma, sarcomas (e.g., fibrosarcoma, myxosarcoma, osteogenic sarcoma, chondrosarcoma, angiosarcoma, endotheliosarcoma, and lymphangiosarcoma), carcinomas (e.g., colon carcinoma, squamous cell carcinoma, basal cell carcinoma, adenocarcinoma, renal cell carcinoma, lung carcinoma, and small cell lung carcinoma), pancreatic cancer, breast cancer, ovarian cancer, prostate cancer, glioma, astrocytoma, neuroblastoma, retinoblastoma, dysplasia, and hyperplasia. The present invention also provides methods and kits for depleting cancerous cells in vitro utilizing C3b(i) specific antibodies. The invention also provides methods and kits for the detection, imaging, and diagnosis of cancer utilizing antibodies specific for C3b(i). Further, the invention provides pharmaceutical compositions comprising antibodies specific for C3b(i).

In accordance with the present invention, antibodies specific for C3b(i) are administered to an animal, preferably a mammal and most preferably a human, to treat, inhibit or prevent cancer or its progression. The antibodies of the present invention comprise monoclonal, polyclonal, bispecific, humanized or chimeric antibodies, single chain antibodies, Fab fragments, and F(ab′) fragments, fragments produced by a Fab expression library, and idiotypic antibodies. In a preferred embodiment, monoclonal antibodies specific for C3b(i) are administered to an animal, preferably a mammal and most preferably a human, to treat, inhibit or prevent cancer. In a particularly preferred embodiment, the monoclonal antibodies are specific for C3b(i) covalently linked to IgM which is bound to the cancer cells. In another preferred embodiment, the monoclonal antibodies are specific for C3b(i) covalently linked to a glycoprotein or glycolipid on the cancer cell. In a specific embodiment, the anti-C3b(i) monoclonal antibodies are conjugated to a therapeutic moiety such as a chemotherapeutic cytotoxin, e.g., a cytostatic or cytocidal agent (e.g., paclitaxol, cytochalasin B or diphtheria toxin), a thrombotic or anti-angiogenic agent or a radioactive label. In another specific embodiment, the valency of anti-C3b(i) monoclonal antibodies is increased to that, for example, of a dimer or an IgM-like pentamer.

In a preferred embodiment, bispecific antibodies which are specific for C3b(i) and an effector cell receptor or antigen are administered to an animal, preferably a mammal and most preferably a human, to treat, inhibit or prevent cancer. The term “effector cell” as used herein refers to a cell which is involved in a cell-mediated immune response, said receptor cells selected from the group, including, but not limited to, monocytes, macrophages, dendritic cells, neutrophils, natural killer cells, lymphocytes and erythrocytes. In one embodiment, anti-C3b(i) heteropolymer constructs (bispecific mAb complexes) bound ex vivo to an effector cell via a cell surface receptor are administered to an animal, preferably a mammal and most preferably a human, to treat, inhibit or prevent cancer. Cell surface receptors include, but are not limited to, CR1, CR2, CR3, CR4, human Fcγ receptors CD16, CD32 and CD64, and the Fc receptor for IgA, CD89. In a preferred embodiment, anti-C3b(i) heteropolymer constructs bound ex vivo to erythrocytes via CR1 are administered to an animal, preferably a mammal and most preferably a human, to treat, inhibit or prevent cancer.

In a preferred embodiment, bispecific diabodies which are antibody fragments specific for C3b(i) and a complement component are administered to an animal, preferably a mammal and most preferably a human, to treat, inhibit or prevent cancer. In accordance with this embodiment, the diabodies are capable of recruiting complement components. In a preferred embodiment, bispecific diabodies which are specific for C3b(i) and C1q are administered to an animal, preferably a mammal and most preferably a human, to treat, inhibit or prevent cancer. Methods of preparing diabodies are taught in U.S. Pat. No. 5,837,242, which is incorporated herein in its entirety.

In one embodiment, IgG and/or IgM antibodies are administered to an animal prior to the administration of antibodies specific for C3b(i). The administration of IgG and/or IgM antibodies facilitates opsonization. In a preferred embodiment, IgM antibodies, preferably normal IgM antibodies from an animal, which contains antibodies to improperly glycosylated cancer cells, are administered to an animal prior to the administration of antibodies specific for C3b(i). In accordance with this embodiment, normal plasma or selectively enriched IgM is administered to an animal, preferably a mammal and most preferably a human. Preferably, the normal plasma or selectively enriched IgM is obtained from an animal of the same species which receives the administration. The normal plasma may or may not be treated with EDTA, citrate or heparin to block the complement pathways. In another embodiment, normal plasma as a source of complement components or recombinant complement components is administered to an animal prior to the administration of antibodies specific for C3b(i). In yet another embodiment, a source of IgM antibodies and complement components (e.g., normal plasma) is administered to an animal to insure efficient opsonization prior to the administration of antibodies specific for C3b(i). In accordance with the invention, the administration of C3b(i) specific antibodies in combination with IgM antibodies and/or complement will initiate a chain reaction which results in increased complement activity and ultimately the killing of cancerous cells.

In a preferred embodiment, the endogenous levels of IgM antibodies and complement components are analyzed to determine whether an animal, preferably a mammal and most preferably a human, requires the administration of IgM antibodies and/or complement components. Standard techniques known to those of skill in the art can be utilized to measure the endogenous levels of IgM antibodies and complement components in an animals sera. For example, the level of IgM antibodies in sera can be determined by titration of the sera against comparable cancer cell lines. Further, the level of complement components and complement activity can be determined by, for example, in vitro tests for the ability to interact with complement proteins, and the ability to lyse target cells opsonized with specific antibodies. (Complement: A Practical Approach, Dodds and Sim, Oxford University Press 1997; Makrides et al., 1992, J. Biol. Chem. 264:24754-24761, Weisman, H. F., et al., 1990, Science, 244:146-151).

In an alternative embodiment of the present invention, IgM antibodies and/or one or more complement components are administered to an animal, preferably a mammal and most preferably a human, without antibodies specific for C3b(i). In accordance with this embodiment, IgM antibodies and/or complement components are administered to an animal to treat, inhibit or prevent cancer.

5.1 IgM Enrichment

In accordance with certain embodiments of the present invention, the levels of IgM antibodies and complement components in the sera or plasma of an animal are measured prior to the administration of anti-C3b(i) antibodies. In one embodiment, animals determined to have low levels of IgM antibodies are administered normal plasma containing IgM antibodies (preferably, IgM antibodies to improperly glycosylated cancer cells). In accordance with this embodiment, the plasma is obtained from an animal of the same species that receives the plasma. In another embodiment, animals determined to have low levels of IgM antibodies are administered plasma enriched for IgM antibodies. In accordance with this embodiment, IgM antibodies are selectively enriched utilizing standard techniques known to those of skill in the art. Such techniques include, but are not limited to, chromatography, centrifugation, and differential solubility. In a particular embodiment of the invention, native or recombinant IgM antibodies known to bind to improperly glycosylated cancer cells are administered to an animal. IgM antibodies to improperly glycosylated cancer cells can be purified utilizing standard protein purification techniques known to those of skill in the art. Such techniques include, but are not limited to, gel purification, chromatography (e.g., ion exchange, affinity, particularly by affinity for the specific antigen after Protein A, and sizing column chromatography), centrifugation, and differential solubility. Recombinant IgM antibodies can be produced utilizing standard techniques known to those of skill in the art.

In a preferred embodiment, IgM antibodies are administered to a subject the same day as the subject is administered antibodies to C3b(i) or antibodies to C3b(i) covalently linked to a second molecule. Preferably, the IgM antibodies are administered to the subject before the antibodies to C3b(i) or antibodies to C3b(i) covalently linked to a second molecule. In yet another preferred embodiment, IgM antibodies are administered to a subject a few hours before administering antibodies to C3b(i) or antibodies to C3b(i) covalently linked to a second molecule.

5.2 Complement Components

In a preferred embodiment, animals determined to have low levels of complement, particularly C3, are infused with normal plasma prior to the administration of anti-C3b(i) antibodies. In accordance with this embodiment, the plasma is obtained from an animal of the same species that receives the plasma. In another preferred embodiment, animals determined to have low levels of complement are administered native or recombinant complement proteins (e.g., C3) prior to the administration of anti-C3b(i) antibodies. In a preferred embodiment, one or more complement components are administered to a subject the same day as the subject is administered antibodies to C3b(i) or antibodies to C3b(i) covalently linked to a second molecule. Preferably, one or more complement components are administered to the subject before the antibodies to C3b(i) or antibodies to C3b(i) covalently linked to a second molecule. In yet another preferred embodiment, one or more complement components are administered to a subject a few hours before administering antibodies to C3b(i) or antibodies to C3b(i) covalently linked to a second molecule.

Complement components, in particular complement component C3, can be purified utilizing standard protein purification techniques known to those of skill in the art. Such techniques include, but are not limited to, gel purification, chromatography (e.g., ion exchange, affinity, particularly by affinity for the specific antigen after Protein A, and sizing column chromatography), centrifugation, and differential solubility. Recombinant complement components (e.g., C3) can be produced utilizing standard techniques known to those of skill in the art. In accordance with the invention, the nucleic acid sequences encoding complement components can be obtained from available sequence databases, e.g., Genbank. Further, the recombinant complement component retains the ability to function in the classical and/or alternative complement pathways.

The nucleotide sequence encoding complement components or a functionally active analogs or fragments or other derivatives thereof (e.g., C3b(i)) can be inserted into an appropriate expression vector, i.e., a vector which contains the necessary elements for the transcription and translation of the inserted protein-coding sequence. For example, the nucleotide sequence encoding human C3 as disclosed in Genbank Accession Numbers NM_{—}000064 and K02765 can be inserted into an appropriate expression vector. In another example, the nucleotide sequence encoding human C1 subcomponents, human C2 or human C2 as disclosed in Genbank Accession Numbers NM_{—}000063, NM_{—}001734, J04080, and AF019413, respectively, can be inserted into an appropriate expression vector. The necessary transcriptional and translational signals can also be supplied by the native complement component genes or its flanking regions. A variety of host-vector systems may be utilized to express the protein-coding sequence. These include but are not limited to mammalian cell systems infected with virus (e.g., vaccinia virus, adenovirus, etc.); insect cell systems infected with virus (e.g., baculovirus); and microorganisms such as yeast containing yeast vectors, or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used. In specific embodiments, the human complement component genes or sequences encoding functionally active portions of the human complement components are expressed.

Any of the methods previously described for the insertion of DNA fragments into a vector may be used to construct expression vectors containing a chimeric gene consisting of appropriate transcriptional and translational control signals and the protein coding sequences. These methods may include in vitro recombinant DNA and synthetic techniques and in vivo recombinants (genetic recombination). Expression of the nucleic acid sequence encoding a complement component or fragments thereof may be regulated by a second nucleic acid sequence so that the complement component or fragments thereof are expressed in a host transformed with the recombinant DNA molecule. For example, expression of complement components (e.g., C3) may be controlled by any promoter or enhancer element known in the art.

Promoters which may be used to control complement component (e.g., C3) gene expression include, but are not limited to, the SV40 early promoter region (Bernoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. USA 78:1441-1445), the regulatory sequences of the metallothionein gene (Brinster et al., 1982, Nature 296:39-42); prokaryotic expression vectors such as the β-lactamase promoter (Villa-Kamaroff et al., 1978, Proc. Natl. Acad. Sci. USA 75:3727-3731), or the tac promoter (DeBoer et al., 1983, Proc. Natl. Acad. Sci. USA 80:21-25); see also “Useful proteins from recombinant bacteria” in Scientific American, 1980, 242:74-94; plant expression vectors comprising the nopaline synthetase promoter region (Herrera-Estrella et al., Nature 303:209-213) or the cauliflower mosaic virus 35S RNA promoter (Gardner et al., 1981, Nucl. Acids Res. 9:2871), and the promoter of the photosynthetic enzyme ribulose biphosphate carboxylase (Herrera-Estrella et al., 1984, Nature 310:115-120); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Omitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells (Mogram et al., 1985, Nature 315 :338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286), and gonadotropic releasing hormone gene control region which is active in the hypothalamus (Mason et al., 1986, Science 234:1372-1378).

In a specific embodiment, a vector is used that comprises a promoter operably linked to complement component (e.g., C3)-encoding nucleic acid, one or more origins of replication, and, optionally, one or more selectable markers (e.g., an antibiotic resistance gene).

Expression vectors containing gene inserts can be identified by three general approaches: (a) nucleic acid hybridization, (b) presence or absence of “marker” gene functions, and (c) expression of inserted sequences. In the first approach, the presence of the complement component gene (e.g., C3) inserted in an expression vector(s) can be detected by nucleic acid hybridization using probes comprising sequences that are homologous to the inserted gene(s). In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of the gene(s) in the vector(s). For example, if the C3 gene is inserted within the marker gene sequence of the vector, recombinants containing the C3 gene insert can be identified by the absence of the marker gene function. In the third approach, recombinant expression vectors can be identified by assaying the gene product expressed by the recombinant. Such assays can be based, for example, on the physical or functional properties of the complement component in in vitro assay systems, e.g., binding of C3 with anti-C3 antibody.

Once a particular recombinant DNA molecule is identified and isolated, several methods known in the art may be used to propagate it. Once a suitable host system and growth conditions are established, recombinant expression vectors can be propagated and prepared in quantity. As previously explained, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered may be controlled. Furthermore, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. For example, expression in a bacterial system can be used to produce an unglycosylated core protein product. Expression in yeast will produce a glycosylated product. Expression in mammalian cells can be used to ensure “native” glycosylation of a heterologous protein. Furthermore, different vector/host expression systems may effect processing reactions to different extents.

For long-term, high-yield production of recombinant proteins, stable expression is preferred. For example, cell lines which stably express the differentially expressed or pathway gene protein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the differentially expressed or pathway gene protein. Such engineered cell lines may be particularly useful in screening and evaluation of compounds that affect the endogenous activity of the differentially expressed or pathway gene protein.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy et al., 1980, Cell 22:817) genes can be employed in tk-, hgprt- or aprt- cells, respectively. Also, antimetabolite resistance can be used as the basis of selection for dhfr, which confers resistance to methotrexate (Wigler et al., 1980, Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin et al., 1981, J. Mol. Biol. 150:1); and hygro, which confers resistance to hygromycin (Santerre et al., 1984, Gene 30:147) genes.

Both cDNA and genomic sequences can be cloned and expressed.

5.3 C3b(i) Specific Antibodies

Antibodies of the invention include, but are not limited to polyclonal, monoclonal, bispecific, humanized or chimeric antibodies, single chain antibodies, Fab fragments and F(ab′) fragments, fragments produced by a Fab expression library, anti-idiotypic (anti-Id) antibodies, and epitope-binding fragments of any of the above. The term “antibody” as used herein refers to immunoglobulin molecules and immunologically active portions of immunoglobulin molecules, i.e., molecules that contain an antigen binding site which specifically binds an antigen. The immunoglobulin molecules of the invention can be of any type (e.g., IgG, IgE, IgM, IgD and IgA), class, or subclass of immunoglobulin molecule.

Polyclonal antibodies which may be used in the methods of the invention are heterogeneous populations of antibody molecules derived from the sera of immunized animals. Various procedures well known in the art may be used for the production of polyclonal antibodies to an antigen-of-interest. For example, for the production of polyclonal antibodies, various host animals can be immunized by injection with an antigen of interest or derivative thereof, including but not limited to rabbits, mice, rats, etc. Various adjuvants may be used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

In a preferred embodiment, the C3b(i) specific antibodies are monoclonal antibodies. Monoclonal antibodies which may be used in the methods of the invention are homogeneous populations of antibodies to a particular antigen (e.g., C3b(i)). A monoclonal antibody (mAb) to an antigen-of-interest can be prepared by using any technique known in the art which provides for the production of antibody molecules by continuous cell lines in culture. These include but are not limited to the hybridoma technique originally described by Kohler and Milstein (1975, Nature 256, 495-497), and the more recent human B cell hybridoma technique (Kozbor et al., 1983, Immunology Today 4, 72), and the EBV-hybridoma technique (Cole et al., 1985, Monoclonal Antibodies and Cancer Therapy, Alan R. Liss, Inc., pp. 77-96). Such antibodies may be of any immunoglobulin class including IgG, IgM, IgE, IgA, IgD and any subclass thereof. The hybridoma producing the mAbs of use in this invention may be cultivated in vitro or in vivo.

The monoclonal antibodies which may be used in the methods of the invention include but are not limited to human monoclonal antibodies or chimeric human-mouse (or other species) monoclonal antibodies. Human monoclonal antibodies may be made by any of numerous techniques known in the art (e.g., Teng et al., 1983, Proc. Natl. Acad. Sci. U.S.A. 80, 7308-7312; Kozbor et al., 1983, Immunology Today 4, 72-79; Olsson et al., 1982, Meth. Enzymol. 92, 3-16).

The invention further provides for the use of bispecific antibodies. Methods for making bispecific antibodies are known in the art. Traditional production of full length bispecific antibodies is based on the coexpression of two immunoglobulin heavy chain-light chain pairs, where the two chains have different specificities (Milstein et al., 1983, Nature 305:537-539). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, published 13 May 1993, and in Traunecker et al., 1991, EMBO J. 10:3655-3659.

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690 published Mar. 3, 1994.

For further details of generating bispecific antibodies see, for example, Suresh et al., Methods in Enzymology, 1986, 121:210. Using such techniques, a bispecific molecule which combines anti-C3b(i) antibody and an antibody specific for an effector cell receptor or antigen can be prepared for use in the treatment or inhibition of disease as defined herein.

The invention provides for the use of functionally active fragments, derivatives or analogs of the anti-C3b(i) immunoglobulin molecules. Functionally active means that the fragment, derivative or analog is able to elicit anti-anti-idiotype antibodies (i.e., tertiary antibodies of Ab3 antibodies) that recognize the same antigen that the antibody from which the fragment, derivative or analog is derived recognized. Specifically, in a preferred embodiment the antigenicity of the idiotype of the immunoglobulin molecule may be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art.

Other embodiments of the invention include fragments of the antibodies of the invention such as, but not limited to, F(ab′)₂ fragments, which contain the variable region, the light chain constant region and the CH1 domain of the heavy chain can be produced by pepsin digestion of the antibody molecule, and Fab fragments, which can be generated by reducing the disulfide bridges of the F(ab′)₂ fragments. The invention also provides heavy chain and light chain dimers of the antibodies of the invention, or any minimal fragment thereof such as Fvs or single chain antibodies (SCAs) (e.g., as described in U.S. Pat. No. 4,946,778; Bird, 1988, Science 242:423-42; Huston et al., 1988, Proc. Natl. Acad. Sci. USA 85:5879-5883; and Ward et al., 1989, Nature 334:544-54), or any other molecule with the same specificity as the antibody of the invention.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, Bio/Techniques 4:214; U.S. Pat. No. 5,225,539; Jones et al., 1986, Nature 321:552-525; Verhoeyan et al. (1988) Science 239:1534; and Beidler et al., 1988, J. Immunol. 141:4053-4060.

Completely human antibodies are particularly desirable for therapeutic treatment of human patients. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Bio/technology 12:899-903).

In other embodiments, the invention provides fusion proteins of the immunoglobulins of the invention (or functionally active fragments thereof), for example in which the immunoglobulin is fused via a covalent bond (e.g., a peptide bond), at either the N-terminus or the C-terminus to an amino acid sequence of another protein (or portion thereof, preferably at least 10, 20 or 50 amino acid portion of the protein) that is not the immunoglobulin. Preferably the immunoglobulin, or fragment thereof, is covalently linked to the other protein at the N-terminus of the constant domain.

The immunoglobulins of the invention include analogs and derivatives that are either modified, i.e, by the covalent attachment of any type of molecule as long as such covalent attachment does not prevent the immunoglobulin from generating an anti-idiotypic response. For example, but not by way of limitation, the derivatives and analogs of the immunoglobulins include those that have been further modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the analog or derivative may contain one or more non-classical amino acids.

5.4 Method of Producing Immunoglobulins

The immunoglobulins of the invention can be produced by any method known in the art for the synthesis of immunoglobulins, in particular, by chemical synthesis or by recombinant expression, and are preferably produced by recombinant expression techniques.

Recombinant expression of the immunoglobulin of the invention, or fragment, derivative or analog thereof, requires construction of a nucleic acid that encodes the immunoglobulin. If the nucleotide sequence of the immunoglobulin is known, a nucleic acid encoding the immunoglobulin may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the immunoglobulin, annealing and ligation of those oligonucleotides, and then amplification of the ligated oligonucleotides by PCR.

Alternatively, the nucleic acid encoding the immunoglobulin may be generated from a nucleic acid encoding the immunoglobulin. If a clone containing the nucleic acid encoding the particular immunoglobulin is not available, but the sequence of the immunoglobulin molecule is known, a nucleic acid encoding the immunoglobulin may be obtained from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

If an immunoglobulin molecule that specifically recognizes a particular antigen is not available (or a source for a cDNA library for cloning a nucleic acid encoding such an immunoglobulin), immunoglobulins specific for a particular antigen may be generated by any method known in the art, for example, by immunizing an animal, such as a rabbit, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the immunoglobulin by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

Once a nucleic acid encoding at least the variable domain of the immunoglobulin molecule is obtained, it may be introduced into a vector containing the nucleotide sequence encoding the constant region of the immunoglobulin molecule (see, e.g., PCT Publication WO 86/05807; PCT Publication WO 89/01036; and U.S. Pat. No. 5,122,464). Vectors containing the complete light or heavy chain for co-expression with the nucleic acid to allow the expression of a complete antibody molecule are also available. Then, the nucleic acid encoding the immunoglobulin can be used to introduce the nucleotide substitutions or deletion necessary to substitute (or delete) the one or more variable region cysteine residues participating in an intrachain disulfide bond with an amino acid residue that does not contain a sulfhydyl group. Such modifications can be carried out by any method known in the art for the introduction of specific mutations or deletions in a nucleotide sequence, for example, but not limited to, chemical mutagenesis, in vitro site directed mutagenesis (Hutchinson et al., 1978, J. Biol. Chem. 253:6551), PCT based methods, etc.

In addition, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., 1988, Science 242:1038-1041).

Antibody fragments which recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments.

Once a nucleic acid encoding the immunoglobulin molecule of the invention has been obtained, the vector for the production of the immunoglobulin molecule may be produced by recombinant DNA technology using techniques well known in the art. Thus, methods for preparing the protein of the invention by expressing nucleic acid containing the immunoglobulin molecule sequences are described herein. Methods which are well known to those skilled in the art can be used to construct expression vectors containing the immunoglobulin molecule coding sequences and appropriate transcriptional and translational control signals. These methods include, for example, in vitro recombinant DNA techniques, synthetic techniques, and in vivo genetic recombination. See, for example, the techniques described in Sambrook et al. (1990, Molecular Cloning, A Laboratory Manual, 2d Ed., Cold Spring Harbor Laboratory, Cold Spring Harbor, N.Y.) and Ausubel et al. (eds., 1998, Current Protocols in Molecular Biology, John Wiley & Sons, NY).

The expression vector is transferred to a host cell by conventional techniques and the transfected cells are then cultured by conventional techniques to produce the immunoglobulin of the invention.

The host cells used to express the recombinant antibody of the invention may be either bacterial cells such as Escherichia coli, or, preferably, eukaryotic cells, especially for the expression of whole recombinant immunoglobulin molecule. In particular, mammalian cells such as Chinese hamster ovary cells (CHO), in conjunction with a vector such as the major intermediate early gene promoter element from human cytomegalovirus is an effective expression system for immunoglobulins (Foecking et al., 198, Gene 45:101; Cockett et al., 1990, Bio/Technology 8:2).

A variety of host-expression vector systems may be utilized to express the immunoglobulin molecules of the invention. Such host-expression systems represent vehicles by which the coding sequences of interest may be produced and subsequently purified, but also represent cells which may, when transformed or transfected with the appropriate nucleotide coding sequences, express the immunoglobulin molecule of the invention in situ. These include but are not limited to microorganisms such as bacteria (e.g., E. coli, B. subtilis) transformed with recombinant bacteriophage DNA, plasmid DNA or cosmid DNA expression vectors containing immunoglobulin coding sequences; yeast (e.g., Saccharomyces, Pichia) transformed with recombinant yeast expression vectors containing immunoglobulin coding sequences; insect cell systems infected with recombinant virus expres-sion vectors (e.g., baculovirus) containing the immunoglobulin coding sequences; plant cell systems infected with recombinant virus expression vectors (e.g., cauliflower mosaic virus, CAMV; tobacco mosaic virus, TMV) or transformed with recombinant plasmid expres-sion vectors (e.g., Ti plasmid) containing immunoglobulin coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the immunoglobulin molecule being expressed. For example, when a large quantity of such a protein is to be produced, for the generation of pharmaceutical compositions of an immunoglobulin molecule, vectors which direct the expression of high levels of fusion protein products that are readily purified may be desirable. Such vectors include, but are not limited, to the E. coli expression vector pUR278 (Ruther et al., 1983, EMBO J. 2:1791), in which the immunoglobulin coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with gluta-thione S-transferase (GST). In general, such fusion proteins are soluble and can easily be purified from lysed cells by adsorption and binding to a matrix glutathione-agarose beads followed by elution in the presence of free gluta-thione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The immunoglobulin coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the immunoglobulin coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the immunoglobulin molecule in infected hosts. (e.g., see Logan & Shenk, 1984, Proc. Natl. Acad. Sci. USA 81:355-359). Specific initiation signals may also be required for efficient translation of inserted immunoglobulin coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, WI38, and in particular, breast cancer cell lines such as, for example, BT483, Hs578T, HTB2, BT20 and T47D, and normal mammary gland cell line such as, for example, CRL7030 and Hs578Bst.

For long-term, high-yield production of recombinant proteins, stable expression is preferred. For example, cell lines which stably express the immunoglobulin molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the immunoglobulin molecule. Such engineered cell lines may be particularly useful in screening and evaluation of compounds that interact directly or indirectly with the immunoglobulin molecule.

A number of selection systems may be used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes can be employed in tk-, hgprt- or aprt- cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):155-215). Methods commonly known in the art of recombinant DNA technology which can be used are described in Ausubel et al. (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1; and hygro, which confers resistance to hygromycin (Santerre et al., 1984, Gene 30:147).

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with recombinant vaccinia virus are loaded onto Ni²⁺ nitriloacetic acid-agarose columns and histidine-tagged proteins are selectively eluted with imidazole-containing buffers.

The expression levels of the immunoglobulin molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing immunoglobulin is amplifiable, increase in the level of inhibitor present in culture of host cell will increase the number of copies of the marker gene. Since the amplified region is associated with the immunoglobulin gene, production of the immunoglobulin will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Proudfoot, 1986, Nature 322:52; Kohler, 1980, Proc. Natl. Acad. Sci. USA 77:2197). The coding sequences for the heavy and light chains may comprise cDNA or genomic DNA.

Once the immunoglobulin molecule of the invention has been recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins.

5.5 Antibody Conjugates

In a preferred embodiment, anti-C3b(i) antibodies or fragments thereof are conjugated to a diagnostic or therapeutic agent. The antibodies can be used diagnostically to, for example, monitor the development or progression of a tumor as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See generally U.S. Pat. No. 4,741,900 for metal ions which can be conjugated to antibodies for use as diagnostics according to the present invention. Examples of suitable enzymes include horseradish peroxidase, alkaline phosphatase, beta-galactosidase, or acetylcholinesterase; examples of suitable prosthetic group complexes include streptavidinfbiotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ¹¹¹In or ⁹⁹TC.

Further, an antibody or fragment thereof may be conjugated to a therapeutic moiety such as a cytotoxin, e.g., a cytostatic or cytocidal agent, a therapeutic agent or a radioactive metal ion. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

In one embodiment, anti-C3b(i) antibodies are conjugated to cobra venom factor. In accordance with the invention, C3b(i) specific antibodies conjugated to cobra venom factor are utilized in vitro to deplete cancerous cells from bone marrow obtained from an animal, preferably a mammal and most preferably a human, with cancer. Methods of conjugating antibodies to cobra venom factor are taught in U.S. Pat. No. 5,773,243.

The conjugates of the invention can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator, a thrombotic agent or an anti-angiogenic agent, e.g., angiostatin or endostatin; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies'84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Alternatively, an antibody can be conjugated to a second antibody to form an antibody heteroconjugate as described by Segal in U.S. Pat. No. 4,676,980.

An antibody with or without a therapeutic moiety conjugated to it can be used as a therapeutic that is administered alone or in combination with cytotoxic factor(s) and/or cytokine(s).

5.6 Depletion of Cancerous Cells In Vitro

The invention provides for methods of depleting cancerous cells from non-cancerous tissues and/or cells in vitro (or ex vivo). In particular, the invention provides for methods of depleting cancerous cells by killing them or by separating them from non-cancerous cells. In one embodiment, anti-C3b(i) antibodies or fragments thereof, alone or in combination with IgM antibodies and/or complement, are combined in vitro with tissues and/or cells obtained from an animal, preferably a mammal and most preferably a human. In a preferred embodiment, anti-C3b(i) antibodies or fragments thereof, alone or in combination with IgM antibodies and/or complement, are combined in vitro with bone marrow obtained from an animal, preferably a mammal and most preferably a human. In accordance with these embodiments, the anti-C3b(i) antibodies can be conjugated to detectable substances (e.g., various enzymes, fluorescent materials, luminescent materials, bioluminescent materials, and radioactive materials) or therapeutic agents (e.g., cytostatic and cytocidal agents), which are disclosed in section 5.5. For example, anti-C3b(i) antibodies may be conjugated to cobra venom factor in order to use enhanced complement activation to lyse the cancer cells. In a preferred embodiment, tissues and/or cells thus depleted of cancerous cells are administered to an animal, preferably a mammal and most preferably a human. In accordance with a specific embodiment, the tissues and/or cells are obtained from an animal with cancer prior to treatment for cancer, and tissues and/or cells depleted of cancerous cells are administered to the animal after the treatment.

In one embodiment, monoclonal antibodies specific for C3b(i) are incubated in vitro with tissues and/or cells obtained from an animal, preferably a mammal and most preferably a human. In a preferred embodiment, the monoclonal antibodies are specific for C3b(i) covalently linked to IgM which is bound to the cancer cells. In another preferred embodiment, the monoclonal antibodies are specific for C3b(i) covalently linked to a glycoprotein or glycolipid on the cancer cells.

In one embodiment IgM monoclonal antibodies specific for C3b(i) are administered to an animal, preferably a mammal and most preferably a human. In accordance with this embodiment, the C3b(i) specific IgM antibodies facilitate complement activation and lysis of the cancer cells.

In a preferred embodiment, bispecific antibodies which are specific for C3b(i) and an effector cell receptor or antigen are incubated in vitro with tissues and/or cells obtained from an animal, preferably a mammal and most preferably a human. In another preferred embodiment, bispecific antibodies which are specific for C3b(i) and a complement component (e.g., C1q) are incubated in vitro with tissues and/or cells obtained from an animal, preferably a mammal and most preferably a human. In a particular embodiment, bispecific diabodies which are antibodies fragments specific for C3b(i) and a complement component (e.g., C1q) are incubated in vitro with tissues and/or cells obtained from an animal, preferably a mammal and most preferably a human. In accordance with this embodiment, the bispecific diabodies facilitate complement mediated lysis of the cancer cells.

Anti-C3b(i) antibodies conjugated to detectable substances can be utilized to sort cancerous cells from non-cancerous cells by methods known to those of skill in the art. In one embodiment, cancerous cells are sorted using a fluorescence activated cell sorter (FACS). Fluorescence activated cell sorting (FACS) is a well-known method for separating particles, including cells, based on the fluorescent properties of the particles (Kamarch, 1987, Methods Enzymol, 151:150-165). Laser excitation of fluorescent moieties in the individual particles results in a small electrical charge allowing electromagnetic separation of positive and negative particles from a mixture.

In one embodiment, cells, particularly bone marrow cells, obtained from an animal, preferably a mammal and most preferably a human, are incubated with fluorescently labeled C3b(i) specific antibodies for a time sufficient to allow the labeled antibodies to bind to the cells, preferably between 10 to 60 minutes. In an alternative embodiment, cells, particularly bone marrow cells, obtained from an animal preferably a mammal and most preferably a human, are incubated with C3b(i) specific antibodies, the cells are washed, and the cells are incubated with a second labeled antibody that recognizes the C3b(i) specific antibodies. In accordance with these embodiments, the cells are washed and processed through the cell sorter, allowing separation of cells that bind both antibodies to be separated from hybrid cells that do not bind both antibodies. FACS sorted particles may be directly deposited into individual wells of 96-well or 384-well plates to facilitate separation.

In another embodiment, magnetic beads can be used to separate cancerous cells from non-cancerous cells. Cancerous cells may be sorted using a magnetic activated cell sorting (MACS) technique, a method for separating particles based on their ability to bind magnetic beads (0.5-100 nm diameter) (Dynal, 1995). A variety of useful modifications can be performed on the magnetic microspheres, including covalent addition of antibody which specifically recognizes C3b(i). A magnetic field is then applied, to physically manipulate the selected beads. The beads are then mixed with the cells to allow binding. Cells are then passed through a magnetic field to separate out cancerous cells.

5.7 Therapeutic Use of Anti-C3b(i) Antibodies

The invention provides for treatment, inhibition or prevention of cancer, including, but not limited to, neoplasms, tumors, metastases, or any disease or disorder characterized by uncontrolled cell growth, by administration of a therapeutic compound. Examples of types of cancer and proliferative disorders include, but are not limited to, leukemia (e.g., myeloblastic, promyelocytic, myelomonocytic, monocytic, erythroleukemia, chronic myelocytic (granulocytic) leukemia, and chronic lymphocytic leukemia), lymphoma (e.g., Hodgkin's disease and non-Hodgkin's disease), fibrosarcoma, myxosarcoma, liposarcoma, chondrosarcoma, osteogenic sarcoma, angiosarcoma, endotheliosarcoma, Ewing's tumor, colon carcinoma, pancreatic cancer, breast cancer, ovarian cancer, prostate cancer, squamous cell carcinoma, basal cell carcinoma, adenocarcinoma, renal cell carcinoma, hepatoma, Wilms' tumor, cervical cancer, uterine cancer, testicular tumor, lung carcinoma, small cell lung carcinoma, bladder carcinoma, epithelial carcinoma, glioma, astrocytoma, oligodendroglioma, melanoma, neuroblastoma, retinoblastoma, dysplasia and hyperplasia. In a particular embodiment, therapeutic compounds of the invention are administered to men with prostate cancer (e.g., prostatitis, benign prostatic hypertrophy, benign prostatic hyperplasia (BPH), prostatic paraganglioma, prostate adenocarcinoma, prostatic intraepithelial neoplasia, prostato-rectal fistulas, and atypical prostatic stromal lesions). The treatment and/or prevention of cancer includes, but is not limited to, alleviating symptoms associated with cancer, the inhibition of the progression of cancer, and the promotion of the regression of cancer. Therapeutic compounds of the invention include, but are not limited to: anti-C3b(i) immunoglobulins, analogs and derivatives (including fragments) thereof (e.g., as described herein) and nucleic acids encoding anti-C3b(i) immunoglobulins, analogs, or derivatives (e.g., as described herein). In one embodiment, commercially available or naturally occurring anti-C3b(i) immunoglobulins, functionally active fragments or derivatives thereof are used in the present invention.

The antibodies of the invention may be administered alone or in combination with other types of cancer treatments (e.g., radiation therapy, chemotherapy, hormonal therapy, immunotherapy and anti-tumor agents). In one embodiment, anti-C3b(i) antibodies are administered to an animal, preferably a mammal and most preferably a human, after surgical resection of cancer. In another embodiment anti-C3b(i) antibodies are administered to an animal, preferably a mammal and most preferably a human, in conjugation with chemotherapy or radiotherapy. In a specific embodiment, men with prostate cancer are administered anti-C3b(i) antibodies in conjugation with androgen ablation therapy.

Generally, administration of products of a species origin or species reactivity (in the case of antibodies) that is the same species as that of the patient is preferred. Thus, in a preferred embodiment, human anti-C3b(i) antibodies, derivatives, analogs, or nucleic acids, are administered to a human patient for therapy or prophylaxis.

5.7.1 Gene Therapy

In a specific embodiment, nucleic acids comprising sequences encoding anti-C3b(i) immunoglobulins or functional derivatives thereof, are administered to treat, inhibit or prevent cancer, by way of gene therapy. Gene therapy refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1-993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215). Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In a preferred aspect, the compound comprises nucleic acid sequences encoding anti-C3b(i) immunoglobulin, said nucleic acid sequences being part of expression vectors that express anti-C3b(i) or fragments or chimeric proteins thereof in a suitable host. In particular, such nucleic acid sequences have promoters operably linked to the anti-C3b(i) coding region, said promoter being inducible or constitutive, and, optionally, tissue-specific. In another particular embodiment, nucleic acid molecules are used in which the anti-C3b(i) coding sequences and any other desired sequences are flanked by regions that promote homologous recombination at a desired site in the genome, thus providing for intrachromosomal expression of the anti-C3b(i) nucleic acids (Koller and Smithies, 1989, Proc. Natl. Acad. Sci. USA 86:8932-8935; Zijlstra et al., 1989, Nature 342:435-438).

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a specific embodiment, viral vectors that contains nucleic acid sequences encoding anti-C3b(i) immunoglobulin are used. For example, a retroviral vector can be used (see Miller et al., 1993, Meth. Enzymol. 217:581-599). These retroviral vectors have been to delete retroviral sequences that are not necessary for packaging of the viral genome and integration into host cell DNA. The nucleic acid sequences encoding the anti-C3b(i) to be used in gene therapy are cloned into one or more vectors, which facilitates delivery of the gene into a patient. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang, et al., 1995, Gene Therapy 2:775-783. In a preferred embodiment, adenovirus vectors are used.

Adeno-associated virus (AAV) has also been proposed for use in gene therapy (Walsh et al., 1993, Proc. Soc. Exp. Biol. Med. 204:289-300; U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, nucleic acid sequences encoding anti-C3b(i) are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598, dated Apr. 28, 1994; Stemple and Anderson, 1992, Cell 71:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

5.8 Demonstration of Therapeutic or Prophylactic Utility

The compounds or pharmaceutical compositions of the invention are preferably tested in vitro, and then in vivo for the desired therapeutic or prophylactic activity, prior to use in humans. For example, in vitro assays to demonstrate the therapeutic or prophylactic utility of a compound or pharmaceutical composition include, the effect of a compound on a cell line, particularly one characteristic of a specific type of cancer, or a patient tissue sample. The effect of the compound or composition on the cell line and/or tissue sample can be determined utilizing techniques known to those of skill in the art including, but not limited to, rosette formation assays and cell lysis assays. In accordance with the invention, in vitro assays which can be used to determine whether administration of a specific compound is indicated, include in vitro cell culture assays in which a patient tissue sample is grown in culture, and exposed to or otherwise administered a compound, and the effect of such compound upon the tissue sample is observed.

5.9 Therapeutic/Prophylactic Administration and Composition

The invention provides methods of treatment, inhibition and prophylaxis by administration to a subject of an effective amount of a compound or pharmaceutical composition of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In another embodiment, the compound or composition can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound or composition can be delivered in a controlled release system. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (1990, Science 249:1527-1533).

In a specific embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment, inhibition and prevention of cancer can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

For antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al., 1997, J. Acquired Immune Deficiency Syndromes and Human Retrovirology 14:193).

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

5.10 Diagnosis and Imaging of Cancer

Labeled antibodies, derivatives and analogs thereof, which specifically bind to C3b(i) can be used for diagnostic purposes to detect, diagnose, or monitor cancer. In a preferred embodiment, cancer is detected in the patient. The patient is an animal, preferably a mammal and most preferably a human.

In an embodiment, diagnosis is carried out by: a) administering to a subject an effective amount of a labeled molecule which specifically binds to C3b(i); b) waiting for a time interval following the administering for permitting the labeled molecule to preferentially concentrate at any cancerous site in the subject (and for unbound labeled molecule to be cleared to background level); c) determining background level; and d) detecting the labeled molecule in the subject, such that detection of labeled molecule above the background level indicates the presence of cancer. Background level can be determined by various methods including, comparing the amount of labeled molecule detected to a standard value previously determined for a particular system.

Depending on several variables, including the type of label used and the mode of administration, the time interval following the administering for permitting the labeled molecule to preferentially concentrate at any cancerous site in the subject and for unbound labeled molecule to be cleared to background level is 6 to 48 hours or 6 to 24 hours or 6 to 12 hours. In another embodiment the time interval following administration is 5 to 20 days r 5 to 10 days.

In an embodiment, monitoring of the cancer is carried out by repeating the method for diagnosing the cancer, for example, one month after initial diagnosis, six months after initial diagnosis, one year after initial diagnosis, etc.

5.10.1 Methods of Detection and Imaging

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include but are not limited to: computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patent using positron emission-tomography. In yet another embodiment, the molecule is labeled with a paramagnetic label and is detected in a patient using magnetic resonance imaging (MRI).

5.11 KITS

The present invention also provides kits that can be used in the above methods. In one embodiment, a kit comprises an antibody to C3b(i) or an antibody to C3b(i) covalently linked to a second molecule in one or more containers. In another embodiment, a kit comprises an antibody to C3b(i) or an antibody to C3b(i) covalently linked to a second molecule and IgM antibody in one or more containers. In another embodiment, a kit comprises an antibody to C3b(i) or an antibody to C3b(i) covalently linked to a second molecule and one or more complement components in one or more containers. In yet another embodiment, a kit comprises an antibody to C3b(i) or an antibody to C3b(i) covalently linked to a second molecule, IgM antibody and one or more complement components in one or more containers.

Preferably, the kits of the present invention further comprise a control antibody which is not specific for C3b(i) or C3b(i) covalently linked to a second molecule. In a specific embodiment, the kits of the present invention contain a labeled C3b(i) specific antibody. In a preferred embodiment, the kits of the invention contain a C3b(i) specific antibody conjugated to a therapeutic agent. In another preferred embodiment, the kits of the present invention contain a C3b(i) specific antibody conjugated to a diagnostic agent. In yet another preferred embodiment, the kits of the present invention contain a purified C3b(i) specific antibody.

### 6. EXAMPLE

### C3b(i) As a Tumor-Specific Antigen

The following example demonstrates that after opsonization of prostate tumor cells, C3b(i) can function as a tumor-specific antigen. Antibodies specific for C3b(i) can be utilized to target tumor cells for the delivery of therapeutic or diagnostic agents, including cytotoxic, chemotherapeutic, immune-enhancing drugs, radioactive compounds, genetic material and immune effector cells.

LNCaP and lineage-derived C4-2 human prostate cancer cell lines were utilized in this example to demonstrate the use of C3b(i) as a target for immunotherapy. The LNCaP/C4-2 progression model recapitulates progression of human neoplastic prostate disease from an androgen-responsive and minimally metastatic (LNCaP cells) to an androgen-refractory (defined as being able to proliferate in castrate hosts) and highly aggressive phenotype (C4-2 subline) (Thalmann et al., 1994, Canc. Res. 54:2577-81; Chung et al., 1996, Urol. Oncol. 2:99-128; Hyytinen et al., 1997, Br. J. Cane. 75:190-5). It shares remarkable similarities with clinical human prostate cancer both in its genotypic and phenotypic changes. Furthermore, the LNCaP/C4-2 progression model has been shown to be a powerful tool for evaluating anti-prostate cancer therapeutic approaches both in vitro and in vivo (Chung et al., 1997, Acta Urol. Jap. 43:815-20), especially with regard to hornone-refractory disease, for which few effective or durable treatment options currently exist (Scher et al., 1994, Sem Oncol 21:630-56).

6.1 Materials and Methods

Cell Lines and Serum Specimens

LNCaP (American Type Culture Collection, Rockville, Md.) and C4-2 (Urocor, Oklahoma City, OK) human prostate cancer cell lines were maintained in T-media with 5% heat inactivated fetal bovine serum (FBS; Gibco, Grand Island, N.Y.). Cultures were maintained at 37° C. in humidified 5% CO₂, split and harvested at 80 to 90% confluence, and treated, if applicable, at 25% confluence. Cells were collected using either phosphate buffered saline (PBS) with 2.5 mM ethylenediitrilotetraacetic acid (EDTA)(Sigma, St. Louis, Mo.) or trypsin (Gibco, Grand Island, N.Y.) diluted 1:10 in phosphate buffered saline (PBS). Samples were then washed twice in PBS by centrifugation at 200×g for 5 min and resuspended at 1×10⁷ cells/ml in PBS with 1% bovine serum albumin (BSA-PBS).

Serum samples were obtained with written informed consent from normal male and female volunteers (University of Virginia, Charlottesville, VA) and from men being followed for prostate disease (University of Virginia and Eastern Virginia Medical School, Norfolk, VA). Prostate disease patients had pathologic documentation of either benign or neoplastic prostate disease. Blood was drawn into SST gel and clot activator Vacutainer tubes (Becton Dickinson, Franklin Lakes, N.J.), held at room temperature for 30 min, and then centrifuged for 20 min at 700×g to obtain serum which was stored at −80° C.

Serum Opsonization of Tumor Cells

Harvested LNCaP and C4-2 tumor cells (1×10⁷ cells/ml in BSA-PBS) were mixed with an equal volume of freshly thawed serum and gently shaken for 20 mm at 37° C. The opsonized cells were washed twice and brought to a final concentration of 1×10⁷ cells/ml in BSA-PBS. Alternative opsonization procedures included addition of 10 mM EDTA to sera to block all complement activation (or use of EDTA-containing plasma), addition of 10 mM ethylene glycol tetraacetic acid (EGTA) and 5 mM Mg (Mg-EGTA) to allow only alternative pathway activation, use of purified IgM (1 mg/ml, Sigma, St. Louis, Mo.), or use of IgM-depleted serum. In this case, IgM was removed from normal human sera (NHS) by incubating 2.5 ml of serum with 1.65 ml (settled volume) anti-human IgM agarose (Sigma, St. Louis, Mo.) on ice for 1 hr with gentle shaking. The depleted serum was separated from the agarose by centrifugation at 1600×g and then stored at −80° C. ELISA determinations (not shown) demonstrated that >90% of the human IgM was specifically removed from the serum by this procedure, but the level of human IgG was reduced by less than 10%.

Monoclonal Antibodies

IgG₁ mAbs 7C12 and 8E11, specific for C3b(i), and IgG₁ mAb HB57, specific for human IgM, have been described (Taylor et al., 1989, J. Immunol. 143: 3626-3631; Tosic et al., 1989, J. Immunol. Methods 120:241-249), and were used in parallel with isotype-matched controls. Radiolabeling with ¹²⁵I or ¹³¹I was performed using the IODOGEN procedure (Fraker et al., 1978, Bioch. Biophys. Res. Comm. 80:849-53; Edberg et al., 1988, J. Immunol. 141:4258-62). IgG₁ mAb 1B4 and IgG₁ mAb 9H3, specific for human complement receptor 1 (CR1), have been previously described (O'Shea et al., 1985, J. Immunol. 134:2580-7; Edberg et al., 1987, J. Immunol. 139:3739-47; Nickells et al., 1998, Clin. Exp. Immunol. 112:27-33). Bispecific mAb complexes (heteropolymers, HP) were prepared by cross-linking each of the anti-CR1 mAbs with one of the two anti-C3b(i) mAbs using previously described methods (Taylor et al., 1997, J. Immunol. 159:4035-44; Segal et al., 1995, Cur. Prot. Immunol. 2:13.1).

Flow Cytometry and Radioimmunoassays

Opsonized cancer cells were probed with fluorescein isothiocyanate (FITC)-labeled goat anti-human IgM Fc5μ (Pierce, Rockford, Ill.), FITC-labeled goat anti-human IgG Fc (Accurate, Westbury, NY), or a cocktail of the anti-C3b(i) mAbs 7C12 and 8E11 (typically, 200 ng of each mAb per 10⁶ cells) followed by a secondary FITC-labeled goat anti-mouse IgG (Sigma, St. Louis, Mo.). All incubations were at 37° C. for 20 min in BSA-PBS. Controls included non-opsonized cells and irrelevant isotype-matched mAbs. In selected cases, cells were stained with propidium iodide (Sigma, St. Louis, Mo., used at a final concentration of 2 ug/ml in BSA-PBS for 5 min, in the dark, on ice) to ascertain IgM or C3b-opsonization of the viable cell populations only (viability was usually >75%). One or two-color fluorescence analysis was performed with CellQuest software on a FACSCalibur (Becton Dickinson, San Jose, Calif.).

Studies of the binding of ¹²⁵I-labeled anti-C3b(i) and anti-human IgM mAbs to cancer cells followed previously published procedures (Taylor et al., 1989, J. Immunol. 143:3626-31; Edberg et al., 1988, J. Immunol. 141:4258-62). Briefly, after opsonization, 1×10⁶ cancer cells were incubated at 37° C. for 20 min with 100 to 2,000 ng of ¹²⁵I-labeled mAbs 7C12, 8E11, HB57 or matched isotype controls. The level of binding of the mAbs to the cancer cells was then determined by centrifuging the sample through oil and measuring radioactive counts in the cell pellets (Ross et al., 1985, J. Immunol. 135:2005-14).

Rosette Experiments

Ten ul of a 50% suspension of human erythrocytes (E) (approximately 5×10⁷ E) in either BSA-PBS or plasma were incubated with 2.5×10⁵ LNCaP or C4-2 cells (either non-opsonized, or serum opsonized as described above) in the presence or absence of 20 ng of an anti-CR1 X anti-C3b(i) heteropolymer. After 30 min at 37° C., the cell mixtures were resuspended in BSA-PBS at a final concentration of 1% E. Light microscopy was used to evaluate the presence and extent of erythrocyte rosettes surrounding the tumor cells.

Immunohistochemistry

Frozen tissue sections (Center for Prostate Disease Research, Washington, D.C., and the Norman Bethune University of Medical Sciences, Jilin, China) were fixed in acetone, treated with 3% hydrogen peroxide, blocked with Super Block (Scytek Laboratories, Logan Utah), and then by Avidin/Biotin Block (Vector Laboratories, Inc., Burlingame, Calif.). Fixed sections were incubated with 4 ug/ml of IgG, mAbs 7C12 and 8EI 1 overnight at 4° C., followed by biotinylated goat anti-mouse IgG and peroxidase-conjugated streptavidin (Biogenex Laboratories, San Ramon, Calif.), and 3-amino-9-ethylcarbozole/H₂O₂ was used as substrate. Mouse IgG, was used as a negative control for staining. The presence and extent of immunohistochemical staining was evaluated by light microscopy.

Radioimmunotherapy Cytotoxicity Studies

The cytotoxic effects of ¹³¹I-labeled anti-C3b(i) (7C12 and 8E11) mAbs on the LNCaP and C4-2 prostate cancer cell lines were evaluated as follows. 1×10⁶ cells of each prostate cancer cell line were opsonized with 25% NHS (diluted in BSA-PBS) or maintained in BSA-PBS at 37° C. for 30 min. After washing twice with PBS, either 2 ug or 200 ng of ¹³¹I-labeled 7C12+8E11 or ¹³¹I-labeled irrelevant mAb (diluted in BSA-PBS) was added to each set of cells and incubated at room temperature for 30 min. The cells were washed twice with PBS, and plated in triplicate in 24-well tissue culture plates (Fisher Scientific, Pittsburgh, Pa.) in T-media +5% FBS at 3×10⁴ cells per well. The plates were then placed in a humidified environment at 37° C. with 5% CO₂. A single media change was performed on day 3. On 5 (LNCaP) and 6 (C4-2) subsequent days, beginning 24 hr after mAb treatment, the triplicate wells were harvested to evaluate cell killing by comparing differences in 3-(4,5-dimethylthiazol-2-yl)-2,5-diphenyl-tetrazolium-bromide (MTT) (Sigma, St. Louis, Mo.) assay results (35).

6.2 Results

C3b(i) and IgM are deposited on prostate cancer cells

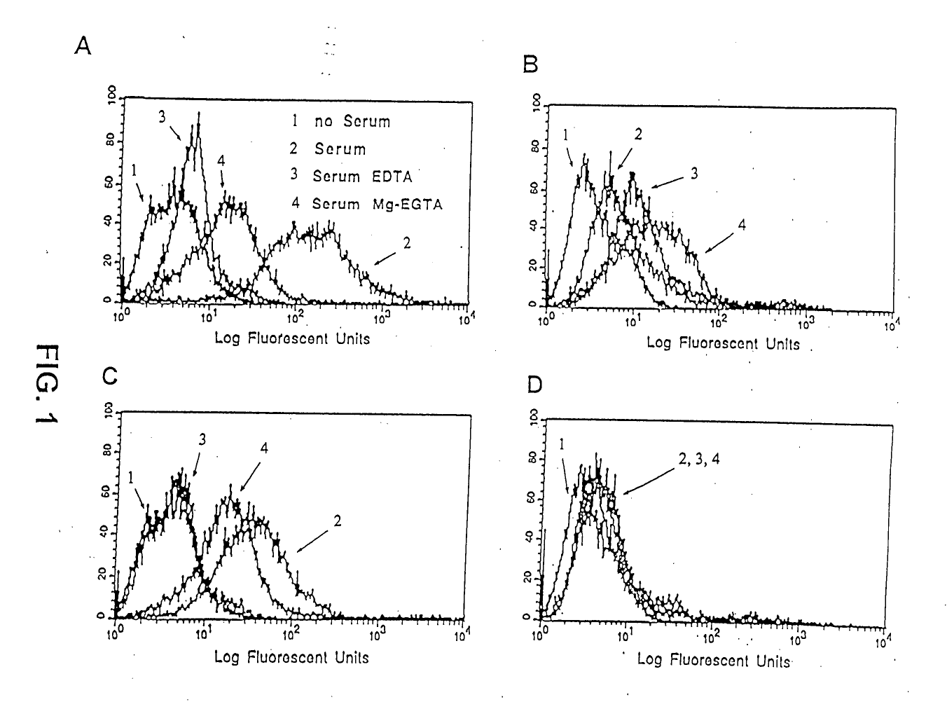

Opsonization of LNCaP and C4-2 prostate cancer cells with normal human serum (“NHS”) results in deposition of substantial amounts of C3b(i) on the cells. In the representative flow cytometry experiment displayed in FIG. 1, the effect of C3b(i) opsonization by NHS on C4-2 cells is shown in the top panel (FIG. 1A). C3b(i) deposition is facilitated by activation of both the classical and alternative complement pathways. However, considerably less C3b(i) is demonstrable when Mg-EGTA, which allows for alternative pathway activation only, is added to the serum. Moreover, opsonization with NHS provides a source of IgM specific for the cancer cells (FIG. 1B). IgM is more readily revealed on the cancer cells when the experiment is conducted under conditions which block the classical pathway of complement activation, as C3b deposition via the classical pathway seems to partially block epitopes on IgM (see Table 1, below). The flow cytometry results also demonstrate that after opsonization with serum from a prostate cancer patient, significantly less C3b(i) and IgM are deposited on the tumor cells (FIGS. 1C and 1D). It is noteworthy, however, that C3b(i) deposition via the alternative pathway (Mg-EGTA-treated serum) is comparable for both the normal and cancer patient serum, suggesting that the alternative pathway of complement activation remains intact in prostate cancer patient serum.

Igm Binding Promotes Robust Cancer Cell Opsonization with C3b(i)

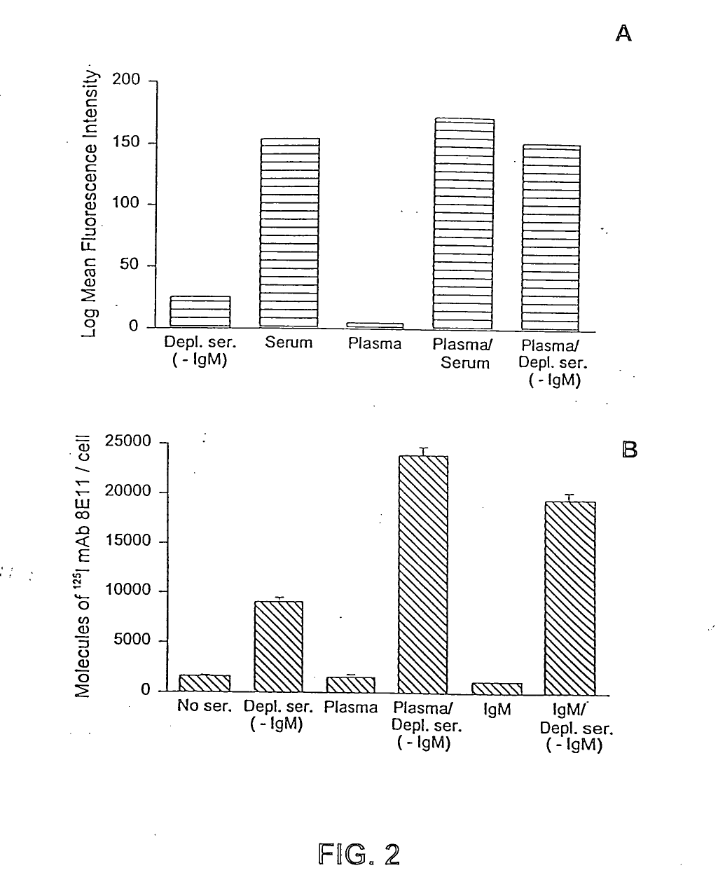

Based on classic studies of the mechanisms of antibody-mediated complement activation (Borsos et al., 1965, Science 150:505-6; Schreiber et al., 1972, J. Clin. Inv. 51:583-9), it was hypothesized that the observed complement activation on the cancer cells as predominantly facilitated by the binding of serum IgM to these cells. To isolate the effects of IgM, affinity chromatography was used to remove IgM from NHS under conditions that preserve the complement activity of the serum. Both RIA and flow cytometry demonstrate that when IgM-depleted serum is used to opsonize LNCap or C4-2 cells, substantially less C3b(i) is deposited on the cancer cells (FIG. 2). Normal levels of C3b(i) deposition can be restored, however, when cancer cells are first incubated with whole normal human plasma containing EDTA, which blocks both classical and alternative complement pathways. The plasma provides a source of human IgM sufficient to allow for robust deposition of C3b(i) on the cancer cells after they are washed and subsequently reacted with the IgM-depleted serum, which serves as a source of complement. RIA analysis further confirms that treatment of the cancer cells with purified IgM followed by treatment with IgM-depleted serum as a complement source also results in enhanced deposition of C3b(i) on the cancer cells (FIG. 2B).

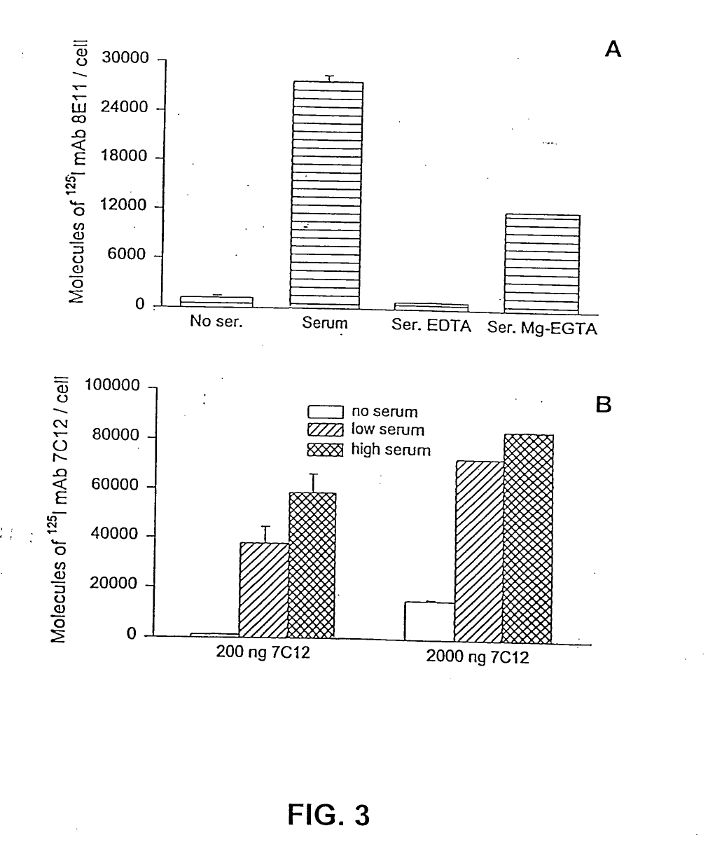

Next, the number of available epitopes on a serum-opsonized cancer cell that can be targeted by anti-C3b(i) mAbs was measured. Dose-response studies were performed under several conditions to estimate the number of C3b(i) sites that are generated on a C4-2 cell after opsonization with NHS in solution phase. The results (displayed in FIG. 3) indicate that the classical pathway of complement activation generates between 20,000 and 70,000 C3b(i) epitopes per C4-2 cell (after correction for background), and that the amount of C3b(i) deposited on the cell is proportional to the quantity of serum used.

The data strongly suggest that natural human IgM binds to surface antigens on cancer cells and facilitates activation of the classical pathway, thus allowing for deposition of large amounts of human C3b(i) on the cells. However, following complement activation and C3b(i) deposition, relatively diminished levels of cancer cell bound IgM can be demonstrated by flow cytometry and RIA (FIG. 1 and Table 1). This is probably due to the fact that once C3b(i) becomes covalently linked to IgM, epitopes on the IgM molecule are obstructed by the C3b(i), thereby preventing the binding of anti-IgM antibodies used for flow cytometry and RIA. Deposition of C3b fragments on human IgM in immune complexes has been documented in several reports (Taylor et al., 1989, J. Immunol. 143:3626-31; Mehta et al., 1986, J. Immunol. 136:1765-71; Thornton et al., 1996, Chin. Exp. Immunol. 104:531-7). Therefore, some C3b(i) is complexed to the IgM on the cancer cell, and it is likely that C3b(i) is also covalently attached to glycoproteins and glycolipids on the cancer cell.

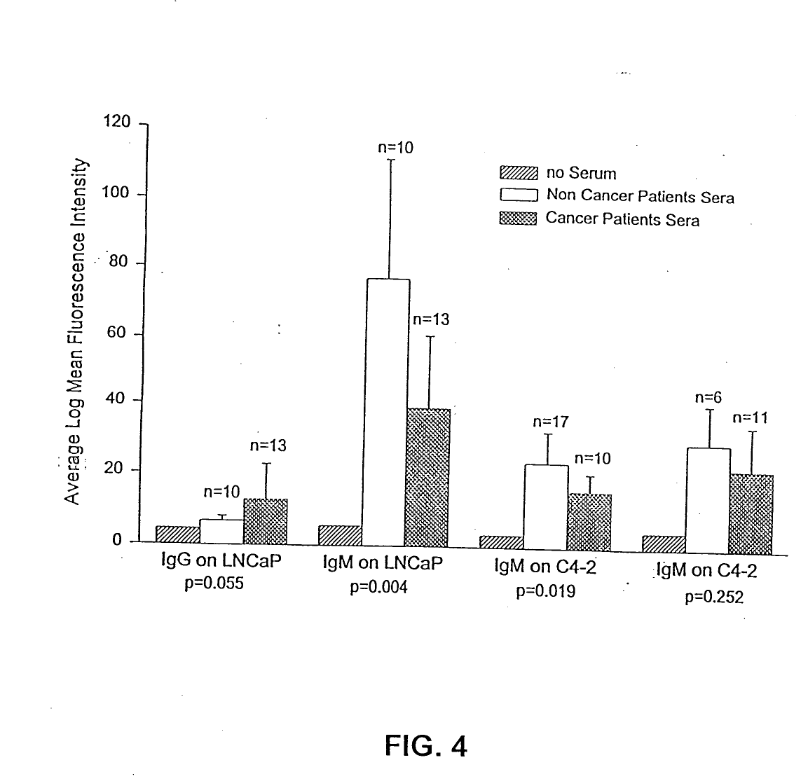

The representative data in FIG. 1 indicates that the serum from a man with prostate cancer is less effective than NHS in depositing C3b(i) on cancer cells. Several studies have previously suggested that the amount of IgM which can bind to cancer cells is reduced in the serum of cancer patients (Desai et al., 1995, J. Immunol. Methods 188:175-85; Seegal et al., 1976, Int. Arch. Allergy App. Immunol. 52:205-11; Higuchi et al., 1980, J. Lab Chin. Immunol. 5:407-18; Gross et al., 1988, Eur. J. Canc. Chin. Oncol. 24:363-7). To independently confirm this hypothesis, sera from a number of normal individuals and men with prostate cancer were surveyed to evaluate differences in the levels of anti-tumor IgM. The experiments were conducted with sera containing 0.01 M EDTA to remove the presumed confounding and blocking effect of C3b(i) in detecting cancer-cell bound IgM. The results, displayed in FIG. 4, indicate that in two of three experiments the level of IgM bound by cancer cells was significantly greater in normal sera when compared to that from prostate cancer patients. The third experiment approaches significance and may have reached it if not for the small number of samples in the control group. In one of the surveys, cancer cell-bound IgG in addition to IgM was assayed. As shown in FIG. 4, little if any IgG in NHS is bound to the cancer cells. However, sera from some of the cancer patients show a notable titer, revealed by the large standard deviation in the patients' group. Although the numbers are too small to draw definitive conclusions, these results do suggest the possibility of an active anti-tumor immune response in some of the cancer patients. Furthermore, those patients with higher anti-tumor IgG titers presented with advanced prostate disease. Such elevated IgG in patients with cancer has previously been reported (Vetvicka et al., 1997, J. Immunol. 159:599-605).

C3b(i) Deposition is Tumor Cell-Specific

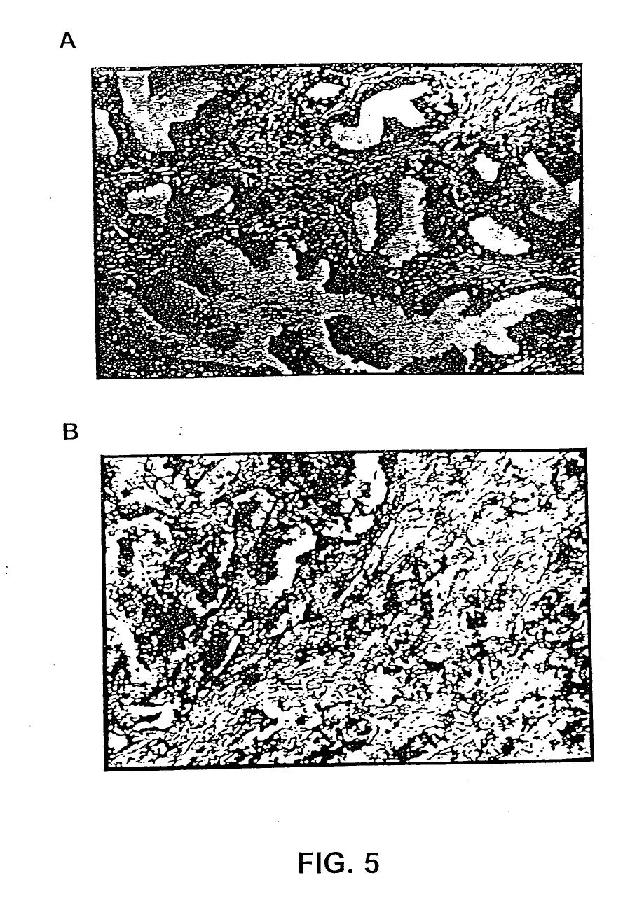

To determine the cancer tissue-specificity of the C3b(i) epitope, a survey of frozen-sectioned prostate tissue specimens with anti-C3b(i) mAbs were immunohistochemically stained. The surgical specimens from two men undergoing transurethral resection for benign prostatic hypertrophy were used as a control. Neither had any immunohistochemical evidence of anti-C3b(i) mAb binding (FIG. 5A). Conversely, of the thirteen specimens from men with prostate cancer, eight (61%) stained positively for anti-C3b(i) mAbs (FIG. 5B). Furthermore, in these eight specimens, only areas of malignancy were stained; regions containing a predominance of benign cells remained negative. In only two specimens was staining of extremely high intensity, implying that although complement is deposited on prostate cancer cells, inherent host complement deposition by itself provides suboptimal opsonization and systemic infusions with IgM (in the form of plasma) from normal donors may be of benefit.

Erythrocytes, Coated With anti-C3b(i) Heteropolymers, Rosette with Opsonized Tumor Cells

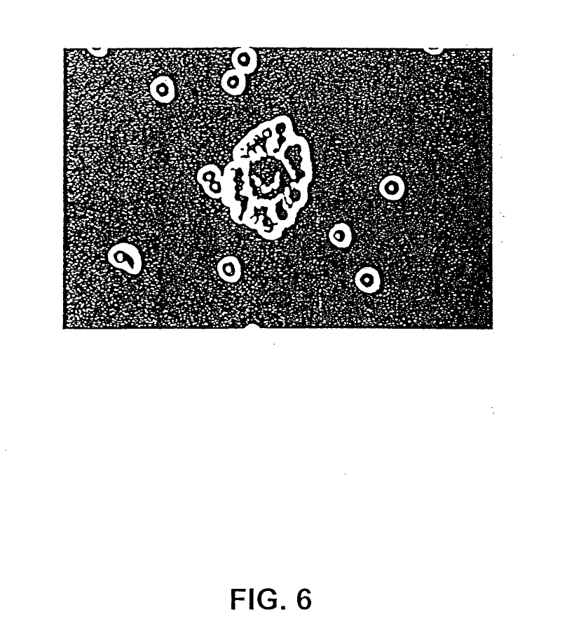

One current application for mAbs in cancer immunotherapy involves the generation of bispecific reagents in which a mAb specific for a cancer cell antigen is cross-linked with a mAb specific for an effector site (e.g., Fc receptors on monocytes/macrophages, granulocytes, or natural killer cells) (Renner et al., 1995, Immunol. Rev. 145:179-209; Clark J I, Alpaugh B K, Weiner L M. Natural killer cell-directed bispecific antibodies. In: Fanger M W, editor. Bispecific Antibodies. ed. Austin: RG Landis Co.; 1995, p. 77-88; Segal D M, Bakacs T, Jost C R, Kurucz I, Sconocchia G, Titus J A. T cell-targeted cytotoxicity. In: Fanger M W, editor. Bispecific Antibodies. ed. Austin: RG Landis Co.; 1995, p. 27-42). In this approach, immune-competent cells can be delivered directly, and specifically, to a tumor via the guidance of the anti-tumor mAb. A prototype for this approach was examined by testing whether human erythrocytes could bind to C3b(i)-opsonized cancer-cells through bispecific mAb complexes (heteropolymers, HP) specific for C3b(i) and the primate erythrocyte complement receptor (CR1). As demonstrated in FIG. 6, rosettes consisting of these erythrocytes completely surrounding the opsonized tumor cells are formed in normal human plasma or in BSA-PBS buffer (not shown). In contrast, in the absence of anti-C3b(i)-specific HP, opsonized tumor cells bind at most only two or three erythrocytes, due to a small amount of CR1-mediated immune adherence (not shown) (Okada et al., 1974, Nature 248:521-25).

Radiolabeled Anti-C3b(i) mAbs Can Kill Prostate Cancer Cells In Vitro

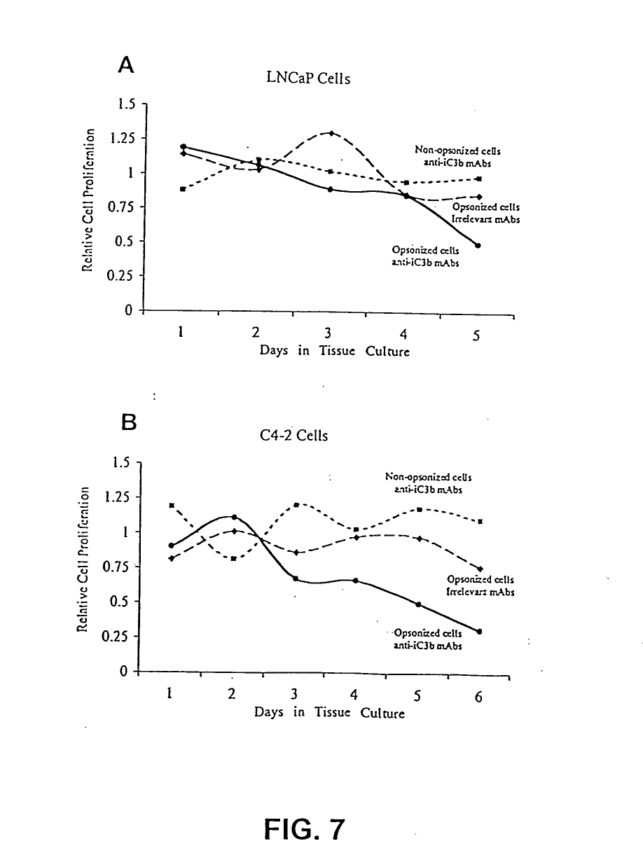

Another application of cancer-specific mAbs involves the coupling of radioactive agents to the mAbs to allow for the imaging or destruction of tumors (Glennie M J, French R R. Targeting drugs, toxins, and radionuclides with bispecific antibodies. In: Fanger M W, editor. Bispecific Antibodies. ed. Austin: RG Landis Co.; 1995, p. 107-20). The potential of this approach was examined by labeling anti-C3b(i) mAbs with ¹³¹I, and then testing their effectiveness in killing cancer cells in culture. After serum opsonization and reaction with the radiolabeled mAbs in solution phase (see Methods), the cells were plated. In all cases the experiments included both serum-opsonized and naive cells, as well as radiolabeled isotype-matched irrelevant mAbs. Although the level of cytotoxicity was modest, progressive killing of serum opsonized LNCaP and C4-2 cells by the ¹³¹I-labeled anti-C3b(i) mAbs over a period of 3 to 6 days was demonstrated (FIG. 7). Cell death was not observed in control cultures consisting of either nonopsonized tumor cells or ¹³¹I-labeled irrelevant mab-treated cells. LNCaP and C4-2 prostate cancer cells opsonized and treated with mAbs after being plated in tissue culture wells demonstrated similar patterns of killing (data not shown).

6.3 Discussion

It has long been recognized that C3b and its fragments can deposit on the surface of cancer cells in patients with tumors (Okada et al., 1974, Nature 248:521-25; Irie et al., 1974, Science 186:454-456.;Vetvicka et al., 1996, J. Clin. Invest. 98:50-61; Vetvicka V et al., 1997, J. Immunol. 159:599-605; Vetvicka et al., 1999, Clin. Exp. Immunol. 115:229-35). This reaction is facilitated by natural IgM. Investigations by Springer and others suggest that the natural IgM repertoire recognizes cancer cell-associated carbohydrate epitopes which are not found on normal tissue (Hakomori et al., 1996, Canc. Res. 56:5309-18; Castronovo et al., 1989, J. Nat. Canc. Inst. 81:212-6; Springer et al., 1984, Science 224:1198-206; Springer et al., 1997, J. Mol. Med. 75;594-602; Desai et al., 1995, J. Immunol. Methods 188:175-85). In fact, several investigators are using carbohydrate epitopes as vaccines to induce an active immune response to certain cancers (Springer, 1984, Science 224:1198-206; Springer, 1997, J. Mol. Med. 75;594-602; Livingston et al., 1997, Canc. Immunol. Immunotherapy 43 :324-30; Zhang et al., 1998, Canc. Res. 58:2844-9). The findings presented herein demonstrate the utility of deposited C3b(i) as a tumor-associated membrane antigen with which to design a general diagnostic and therapeutic modality.

Large amounts of C3b(i) have been shown to specifically deposit on cancer cells after opsonization with NHS (FIGS. 1, 2 and 3). As indicated in FIGS. 1 and 4, the level of the presumably protective IgM is often reduced in cancer patients, including those with breast tumors (Desai et al., 1995, J. Immunol. Methods 188:175-85; Seegal et al., 1976, Int. Arch. Allergy App. Immunol. 52:205-11; Higuchi et al., 1980, J. Lab Chin. Immunol. 5:407-18; Gross et al., 1988, Eur. J. Canc. Chin. Oncol. 24:363-7). Therefore, the infusion of normal human plasma in some cancer patients will help to restore or enhance C3b(i) opsonization of tumor sites accessible to the bloodstream. However, even if normal human plasma deposits a large quantity of C3b(i) on the cancer cell surface, it is unlikely that this action alone will be sufficient to eradicate a tumor, since cancer cells often express high levels of complement control proteins (Gorter et al., 1996, Lab. Invest. 74:1039-49; Maenpaa et al., 1996, Am J Path 148:1139-52; Li et al., 1997, Int. J. Canc. 71:1049-55). For example, the expression of CD59 (“protectin”) by cancer cells blocks the action of the membrane attack complex which might otherwise lyse the cancer cell. The results presented herein demonstrate that one approach to treating cancer is to infuse a patient with normal human plasma (to supply IgM and, if necessary, complement) and to then deliver systemically anti-neoplastic agents to the cancer cells by conjugating the agents to anti-C3b(i) mAbs, which would circulate through the body and home to sites of opsonized tumor cells.

To ensure that a sufficient quantity of therapeutic agent is delivered in close proximity to the tumor cell, mAb-based immunotherapy for cancer requires a very high level of selective and high avidity binding of the mAb to the tumor. The results indicate that at least 20,000 C3b(i) epitopes are available on opsonized prostate cancer cells and, based on the in vitro killing studies, this level of cancer-associated antigen should be sufficient for specific targeting of the cancer cell, enabling the delivery of abundant therapeutic agent.

Tumor tissue-specific delivery of therapeutic agents is crucial to avoid undesirable injury to healthy tissue. In the case of C3b(i) as a target, it is important that complement activation be limited to tumor cells. Except for a few relatively rare disease conditions (Rosse et al., 1995, Blood 86;3277-86; Morgan B P. Complement: clinical aspects and relevance to disease. ed. London: Harcourt Brace Jovanovich; 1990.), the complement system is highly regulated and C3b(i) is not deposited on normal tissue. Moreover, C3b(i) deposition has been shown to be confined to areas of malignancy in human prostate tissue specimens, and is absent in benign (FIG. 5A) and hyperplastic regions (not shown). These data confirm earlier studies on breast cancer, which established a similar tumor tissue-specific pattern of opsonization (Vetvicka et al., 1997, J. Immunol. 159:599-605; Howard et al., 1979, Cancer 43:2279-87; Niculescu et al., 1992, Am. J. Path. 140:1039-43).

Due to normal turnover, a small fraction of circulating C3 expresses antigenic epitopes similar to C3b(i), and this endogenous C3b(i)-like molecule might block the action of the anti-C3b(i) mAbs (Mollnes et al., 1987, J. Immunol. Methods 101;201-7; Petronis et al., 1998, Clin. Nuc. Med. 23:672-7). However, use of bispecific mAb complexes specific for an effector cell receptor and C3b(i) should allow for multivalent and therefore high avidity interaction of the effector cell with the opsonized cancer cell, thus facilitating robust binding in plasma (FIG. 6). Alternatively, C3b(i) covalently linked to IgM on cancer cells should contain unique and specific antigenic determinants against which new mAbs can be developed. Indirect evidence has been presented in Table 1 that indicates that C3b(i) will covalently bind to and block epitopes on human IgM bound to the cancer cell. Therefore, unique and specific neoepitopes are generated as a consequence of this covalent binding reaction which can be used to produce appropriate mAbs to target these sites on cancer cells.

Once the tumor cells are opsonized, anti-C3b(i) mAbs coupled with toxic agents or radioisotopes can be administered to individuals. The potential use of this approach is illustrated in FIG. 7. When LNCaP and C4-2 cells were treated with ¹³¹I-labeled specific anti-C3b(i) mAbs, only those cells that had been opsonized with NHS prior to treatment with the ¹³¹I-anti-C3b(i) mAbs were killed (FIG. 7). This approach can also be utilized for diagnostic imaging purposes, similar to the PROSTASCINT™ scan, when tumor cell deposits are effectively opsonized and then targeted with anti-C3b(i) mAb-conjugated compounds (Petronis et al., 1998, Clin. Nuc. Med. 23:672-7).

Another application is the use of anti-C3b(i) mAbs in bispecific mAb complexes bound to either erythrocytes or immune effector cells (Renner et al., 1995, Immunol. Rev. 145:179-209; Clark J I, Alpaugh B K, Weiner L M. Natural killer cell-directed bispecific antibodies. In: Fanger M W, editor. Bispecific Antibodies. ed. Austin: RG Landis Co.; 1995, p. 77-88; Segal D M, Bakacs T, Jost C R, Kurucz I, Sconocchia G, Titus J A. T cell-targeted cytotoxicity. In: Fanger M W, editor. Bispecific Antibodies. ed. Austin: RG Landis Co.; 1995, p. 27-42; DeGast et al., 1997, Canc. Immunol. Immunotherapy 45:121-3; Taylor et al., 1997, Canc. Immunol. Immunotherapy 45:152-5). The potential use of this approach is illustrated by the rosetting data in FIG. 6. In the presence of anti-C3b(i) crosslinked with anti-CR1 HP, erythrocytes completely encircled those prostate tumor cells opsonized with human serum.

The results herein demonstrate that while opsonization with normal human serum results in the deposition of large amounts of IgM and C3b(i) on prostate cancer cells, opsonization with sera from most men with prostate cancer leads to substantially diminished levels of cell-associated IgM and C3b(i). This deficiency can be restored by the infusion of normal plasma as a source of human IgM which will ultimately allow for the opsonization of cancer cells with C3b(i). These opsonized cells will therefore present unique and specific antigenic determinants for targeting by appropriate C3b(i) mAbs.

The present invention is not to be limited in scope by the exemplified embodiments, which are intended as illustrations of single aspects of the invention. Indeed, various modifications of the invention in addition to those shown and described herein will become apparent to those skilled in the art from the foregoing description and accompanying drawings. Such modifications are intended to fall within the scope of the appended claims.

All publications cited herein are incorporated by reference in their entirety.

TABLE 1
C3b(i) deposition on C4-2 cells by sera from two different
normal donors partially blocks the detection of human IgM
by both flow cytometry and RIA.

| | | | Number of bound ¹²⁵I-mAbs | |
|---|---|---|---|---|
| | Log mean | | | Anti-human |
| | fluorescence intensity^{a} | | Anti-C3b(i) | IgM |
| | | Anti-human | mAb 8E11 | mAb HB57 |
| | Anti-C3b(i) | IgM | (mean ± S.D.) | (mean ± S.D.) |
| No serum | 7.5 | 4.7 | 1,200 ± 270^{b} | 850 ± 30 |
| Serum | 266 | 14.8 | 27,700 ± 70^{b} | 3,100 ± 60 |
| Serum + | 9.1 | 36.8 | 880 ± 100^{b} | 7,900 ± 30 |
| EDTA | | | | |
| Serum + | 55.6 | 29.3 | 12,000 ± 50^{b} | 7,300 ± 330 |
| Mg-EGTA | | | | |

^{a}same data presented in FIG. 1
^{b}same data presented in FIG. 3A

1-13. (cancelled)

14. A pharmaceutical composition comprising an antibody to C3b(i) conjugated to a therapeutic agent, in an amount effective to inhibit or prevent cancer in a subject.

15. (cancelled)

16. The pharmaceutical composition of claim 14 in which the antibody is specific for C3b(i) covalently linked to IgM on cancer cells.

17. The pharmaceutical composition of claim 14 in which the antibody is specific for C3b(i) covalently linked to glycoproteins or glycolipids on cancer cells.

18. The pharmaceutical composition of claim 14, wherein the antibody is a bispecific antibody which is specific for C3b(i) and an effector cell receptor or antigen.

19-40. (cancelled)

41. The pharmaceutical composition of claim 14 in which the antibody is purified.

42. The pharmaceutical composition of claim 14 or 41 further comprising a pharmaceutically acceptable carrier.

43. A kit comprising, in one or more containers, an antibody to C3b(i) conjugated to a therapeutic agent.

44. The kit of claim 43 further comprising IgM antibody.

45. The kit of claim 43 or 44 further comprising one or more complement components.

46-47. (cancelled)

48. The pharmaceutical composition of claim 14, wherein the antibody is a monoclonal antibody.

49. The pharmaceutical composition of claim 14, wherein the antibody is a humanized antibody.

50. The pharmaceutical composition of claim 14, wherein the therapeutic agent is a radioactive agent.

51. The pharmaceutical composition of claim 14, wherein the therapeutic agent is a cytotoxin.

52. The pharmaceutical composition of claim 14, wherein the therapeutic agent is selected from the group consisting of paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin.

53. The pharmaceutical composition of claim 14, wherein the therapeutic agent is cobra venom factor.

54. The pharmaceutical composition of claim 14, wherein the therapeutic agent is abrin, ricin A, pseudomonas exotoxin, or diphtheria toxin.

55. The pharmaceutical composition of any one of claim 16-18 or 48-54, wherein the antibody is purified.
